(19) United States
(12) Patent Application Publication
(10) Pub. No.: US 2006/0134739 A1
(43) Pub. Date: Jun. 22, 2006

(54) Synthesis of proteins by cell-free protein expression

(76) Inventor: Deb K. Chatterjee, Potomac, MD (US)

Correspondence Address: WOODCOCK WASHBURN LLP, ONE LIBERTY PLACE - 46TH FLOOR, PHILADELPHIA, PA, 19103, US

(21) Appl. No.: 10/548,634

(22) PCT Filed: Mar. 10, 2004
(86) PCT No.: PCT/US04/07449

§ 371 Date: Sep. 9, 2005

Related U.S. Application Data
(60) Provisional application No. 60/454,013, filed on Mar. 11, 2003.

(51) Int. Cl. C12P 21/06; C12N 1/21

(52) U.S. Cl. 435/69.1; 435/252.33

(57) ABSTRACT

In Vitro peptide/protein or biological macromolecule synthesis systems, and methods and kits thereof improve efficiency of in vitro protein or biological macromolecule synthesis and related compositions.

### BACKGROUND

In vitro protein synthesis has advantages in specifically producing the desired protein without unnecessarily producing undesired proteins which are normally required for cell maintenance in vivo. Since the cellular maintenance is not required in the cell-free system, almost all of the energy possibly could be channeled to produce protein. Cell-free systems are very popular because there are standard protocols available for their preparation and because they are commercially available from a number of sources.

Because it is essentially free from cellular regulation of gene expression, in vitro protein synthesis has advantages in the production of proteins that would normally be cytotoxic, unstable, or insoluble. The over-production of protein beyond a predetermined concentration can be difficult to obtain in vivo, because the expression levels are often regulated by the concentration of product. In the process of protein isolation and purification, many kinds of protein are insoluble or unstable, and are either degraded by intracellular proteases or aggregate in inclusion bodies, so that the yields are low. In vitro protein synthesis circumvents many of these problems. In addition, simultaneous and rapid in vitro expression of various proteins in a multiplexed configuration can provide a valuable tool for development of combinatorial arrays for research, and for screening of proteins. In addition, various kinds of non-naturally occurring amino acids can be efficiently incorporated into proteins for specific purposes (Noren et al, 1989, Science 44: 182-188). However, despite all its promising aspects, the in vitro system has not been widely accepted as a practical alternative, due to a number of difficulties, in part, relating to the short reaction period, which causes a poor yield of protein synthesis.

Cell-free transcription translation system (CFTT) is an important tool for analysis of proteins. The availability of complete genome sequences provides a wealth of information on the molecular structure and organization of a myriad of genes and open reading frames whose functions are not known or are only poorly understood. Thus, the utility of CFTT and more generally, protein synthesis in vitro, will continue to be important in the future for rapid and efficient protein synthesis and functional analysis.

Cell extracts have been developed which allowed the synthesis of protein in vitro from a purified mRNA transcript. To date, several systems have become available for the study of protein synthesis and for the study of RNA structure and function. To synthesize a protein of interest, a translation extract must be “programmed” with an mRNA corresponding to the gene and protein under investigation. The mRNA can be produced from DNA, or the mRNA can be added exogenously in purified form. Such mRNA templates have been purified from natural sources or have been prepared synthetically from cloned DNA using bacteriophage RNA polymerases in an in vitro reaction.

The cell extracts used for coupled in vitro transcription and translation techniques contain all the components necessary for both transcription (to synthesize mRNA) and for translation (to synthesize protein) in a single system. In such a system, the input template is DNA, which is normally much easier to obtain than RNA and much more readily manipulable. An early coupled transcription/translation system was based on a bacterial extract (Lederman and Zubay, 1967, Biochim. Biophys. Acta. 149: 253). Since prokaryotes normally carry out a coupled reaction within their cytoplasm, this bacterial based system closely reflected the in vivo process. This general system has seen widespread use for the study of prokaryotic genes. However, this general bacterial system is generally not useful for eukaryotic genes, due to its inefficiency and relatively high nuclease content, DNase or RNase with various substrate requirements. A further problem has been the inefficiency of the energy regeneration system which leads to low yields of protein production in the in vitro system.

Although the coupled transcription translation systems are useful for many proteins, translation efficiencies vary widely depending on the type of DNA template which is used (e.g., supercoiled plasmid DNA or linear DNA). In addition, the amount of mRNA synthesized in a coupled reaction is difficult to control under most coupled conditions. Since efficiency and fidelity of translation are dependent upon the amount of mRNA added to and present during the reaction, a possible explanation for the undesirable variability of results obtained using these coupled systems, in which the reactions occur simultaneously, is that transcription and translation are not consistent between various templates under the conventional reaction conditions.

A number of subsequent modifications have been made to improve the CFTT system. (see e.g., Kim et al, 1996, Eur. J. Biochem. 239: 881-886; Patnaik and Swartz, 1998, Biotechniques. 24: 862-868; and Kim and Swartz, 1999, Biotech. and Bioeng. 66: 180-188.) One of the main problems of the conventional CFTT systems is that these systems do not produce sufficient quantities of protein for extensive analysis of protein(s) of interest. The inefficient protein synthesis resulting from conventional synthesis systems can also be attributed, in part, to factors relating to the maintenance of amino acid and energy supplies, stability of the DNA template for transcription, stability of mRNA for translation, and maintenance of energy regeneration systems to support the synthesis reaction.

Two methods have been discussed for replenishing ATP as an energy source necessary for a CFTT system. In one method, the CFTT system utilizes phosphoenolpyruvate (PEP) and an enzyme, pyruvate kinase, to regenerate ATP from ADP and is optimized by adding combinations of amino acids to the system, for example, arginine; tryptophan and cysteine; aspartic acid and asparagines; and alanine. In a second method, pyruvate and an enzyme, pyruvate oxidase, are used to regenerate ATP from ADP. Both energy sources and amino acids are depleted in these systems irrespective of protein synthesis (PCT International Application WO/0055353; Kim and Choi, 2000, J. Biotech. 84: 27-32). Protein synthesis could be restored by a second addition of amino acids and the energy source. Improved efficiency of protein synthesis in the PEP system was achieved using an E. coli S30 extract of lower phosphatase activity, higher cysteine concentration, and an inhibitor of glutathione synthesis pathway.

In some cases, E. coli cell-free protein synthesis systems use phosphoenolpyruvate (PEP) as an ATP regeneration substrate, to which acetyl phosphate and creatine phosphate are also added in combination with PEP. The PEP is converted to pyruvate by pyruvate-kinase, and as a result, ATP is synthesized and used for protein synthesis. However, the protein synthesis in the cell-free systems ceased abruptly within about 30 min. Although, the reasons for the early cessation of protein synthesis have been unclear, it has been shown that unproductive depletion of PEP can be one of the possible reasons. In fact, almost 70% depletion of PEP was shown to be dephosphorylated after 30 min by phosphatases present in the S30 extract (Kim and Swartz, 1999, Biotech. Bioeng. 66: 180-188). The authors also indicated similar fate for acetyl phosphate and creatine phosphate. Thus, this expression-independent consumption/depletion of PEP led to the production of low amount of protein synthesis due to unavailability of PEP to generate ATP in a sustained manner.

A further method for regeneration of ATP as an energy source in a CFTT system requires addition of glucose, glucose-6-phosphate, phosphoenolpyruvate (PEP), or pyruvate, as an energy source, in combination with NADH cofactor. Additional cofactors, such as NADH in combination with acetyl CoA or coenzyme A, are required and add considerable expense to the CFTT system. PCT International Application WO 02/40497; U.S. Pat. No. 6,337,191.

Although these methods can be useful for regenerating ATP, inefficient use of the ATP results in reduced protein yields. For example, phosphatases can hydrolyze the ATP, thus making ATP energy unavailable for the desired CFTT system. The alkaline phosphatase of E. coli is one known phosphatase that can hydrolyze ATP (see, for example, Hyone-Myong Eun, Enzymology Primer for Recombinant DNA Technology, Academic Press, pages 307-333). Helicase enzymes also hydrolyze ATP. Therefore, the ATP that is being made from the energy generating system with a goal of driving protein synthesis can potentially be wasted by active phosphatases, such as alkaline or acid phosphatases, with a resultant reduction of protein synthesis.

It has been shown in a wheat germ cell-free system that immunodepletion of phosphatase improved protein synthesis by reducing ATP and GTP hydrolysis (Kawarasaki et al, 1998, J. Biotech. 61: 199-208). It has also been suggested that phosphoenolpyruvate (PEP), the substrate generally used for regeneration of ATP, is also degraded by phosphatases, thus limiting protein synthesis (Kim and Swartz, 1999, Biotech. and Bioeng. 66: 180-188; Swartz and Kim, U.S. Pat. No. 6,168,931; Kim and Choi, 2000, J. Biotech. 84: 27-32).

In view of the present state of the art, there is a need to develop an improved CFTT system that will enhance the production of protein(s). The present invention meets this need by virtue of providing compositions and methods for stabilizing or maintaining template DNA, by stabilizing or maintaining mRNA including mRNA derived from the template(s), by conserving energy to be used for synthesis and/or by providing sufficient energy regenerating substrates to provide the energy necessary for efficient protein synthesis from the templates.

### SUMMARY

The present invention relates to in vitro synthesis, more specifically to in vitro peptide/protein or biological macromolecules synthesis systems, and to methods and kits that improve efficiency of such in vitro synthesis and related compositions. More specifically, the present systems, compositions, methods and kits improve efficiency of protein or biological macromolecules synthesis by providing an improved energy supply or energy regeneration system for synthesis and/or by maintaining the nucleic acid template(s) for synthesizing proteins or biological macromolecules for a more efficient and extended synthesis reaction. The present systems, compositions, methods and kits can be used in any type of in vitro protein synthesis system, including coupled transcription/translation systems and uncoupled translation systems. In vitro protein or biological macromolecules synthesis systems comprise at least one cell extract, at least one energy source, and at least one nucleic acid template.

In one embodiment, an in vitro protein or biological macromolecules synthesis system includes one or more energy sources providing chemical energy for protein or biological macromolecules synthesis, wherein at least one of the energy sources is a glycolytic intermediate. In a further aspect, the glycolytic intermediate is selected from 3-phosphoglycerate, 2-phosphoglycerate, 2,3-diphosphoglycerate, or 1,3-diphosphoglycerate. In a detailed aspect, the glycolytic intermediate is 3-phosphoglycerate. In a further aspect of the in vitro protein or biological macromolecules synthesis system, the glycolytic intermediate provides chemical energy for protein or biological macromolecules synthesis without addition of an exogenous enzymatic cofactor. In a further aspect, the exogenous enzymatic cofactor includes, but is not limited to, thiamine pyrophosphate, FAD⁺, FADH, NAD⁺, NADH, NADP⁺, or NADPH. In a detailed aspect, the exogenous enzymatic cofactor is NAD⁺ or NADH.

In an aspect of the in vitro protein or biological macromolecules synthesis system, the glycolytic intermediate provides chemical energy for protein or biological macromolecules synthesis without addition of an exogenous enzyme. In a detailed aspect, the exogenous enzymes include, but are not limited to, pyruvate kinase, acetate kinase, pyruvate oxidase, creatine kinase, or creatine phosphokinase

In a further aspect, the glycolytic intermediate provides chemical energy for protein or biological macromolecules synthesis without addition of an exogenous enzymatic cofactor, for example, NAD⁺ or NADH. In a further aspect, the in vitro protein or biological macromolecules synthesis system includes a glycolytic intermediate as an energy source, in combination with adenosine triphosphate and cysteine. In a detailed aspect of the in vitro protein or biological macromolecules synthesis system, the glycolytic intermediate provides chemical energy for protein or biological macromolecules synthesis without addition of an exogenous enzymatic cofactor or an exogenous enzyme.

Some such in vitro synthesis system include at least one nucleic acid template selected from a DNA template and an RNA template. In one aspect the in vitro synthesis system includes at least one DNA template, and the in vitro synthesis system is an in vitro transcription/translation system or an in vitro protein synthesis system. The product of the in vitro transcription/translation system is a biological macromolecule selected from polypeptide, protein, or ribonucleic acid. In a further aspect the in vitro synthesis system includes at least one DNA template, and the in vitro synthesis system is an in vitro transcription system or an in vitro biological macromolecules synthesis system. The product of the in vitro transcription system is a ribonucleic acid (RNA), which can be mRNA, rRNA, tRNA, antisense RNA, ribozymes or interfering RNA (RNAi).

Some such in vitro protein or biological macromolecules synthesis systems include a cellular extract to provide components selected from enzymes, ribosomes, transcription factors, translation factors or co-factors. The in vitro protein or biological macromolecule synthesis system further includes a cellular extract or cellular components for E. coli rare t-RNAs selected from tRNAs for amino acids arginine, proline, glycine, leucine or isoleucine. In a further detailed aspect, the in vitro protein or biological macromolecules synthesis system includes a cellular extract to provide cellular components selected from lipids, cholesterol, or membranes.

In a further aspect, the in vitro protein or biological macromolecules synthesis system utilizes two or more energy sources which provide chemical energy for synthesis. The two or more energy sources are selected from at least one energy source from 3-phosphoglycerate, 2-phosphoglycerate, 2,3-diphosphoglycerate, or 1,3-diphosphoglycerate. The two or more energy sources include, but are not limited to, pyruvate, phosphoenolpyruvate (PEP), carbamoyl phosphate, acetyl phosphate, creatine phosphate, phosphopyruvate, 3-phosphoglycerate, 2-phosphoglycerate, triose phosphate, 2,3-diphosphoglycerate, 1,3-diphosphoglycerate, glyceraldehyde-3-phosphate, fructose-1,6-diphosphate, fructose-6-phosphate, glucose-1-phosphate, glucose-6-phosphate, or glucose.

In another embodiment, an in vitro protein or biological macromolecules synthesis system includes two or more energy sources providing chemical energy for protein or biological macromolecules synthesis, wherein at least one of the energy sources is a glycolytic intermediate and a second energy source is a tricarboxylic acid (TCA) cycle intermediate. In a further aspect, the glycolytic intermediate is selected from 3-phosphoglycerate, 2-phosphoglycerate, 2,3-diphosphoglycerate, or 1,3-diphosphoglycerate. In a detailed aspect, the glycolytic intermediate is 3-phosphoglycerate. In a further aspect, the TCA cycle intermediate is selected from citrate, isocitrate, malate, oxaloacetate, α-ketoglutarate, succinate or fumarate. In a detailed aspect, the TCA cycle intermediate is citrate. In a further aspect of the in vitro protein or biological macromolecules synthesis system, the glycolytic intermediate and the TCA cycle intermediate further comprise an exogenous enzymatic cofactor to provide chemical energy for protein or biological macromolecules synthesis. In a further aspect, the exogenous enzymatic cofactor includes, but is not limited to, thiamine pyrophosphate, FAD⁺, FADH, NAD⁺, NADH, NADP⁺, or NADPH. In a detailed aspect, when citrate is used as the energy source, the exogenous enzymatic cofactor can be NAD⁺ or NADH. In a further detailed aspect, when succinate is used as the energy source, the exogenous enzymatic cofactor can be FAD⁺ or FADH.

Some such in vitro protein or biological macromolecules synthesis systems can include a cell extract having a reduced activity of an enzyme or other cellular component. The extract can be from a cell having reduced activity of at least one enzyme that catalyzes hydrolysis of high-energy phosphate bonds, or the extract can be from a cell having reduced activity of at least one enzyme that catalyzes hydrolysis or formation of phosphodiester bonds. In a further embodiment, the in vitro protein or biological macromolecules synthesis system can further include a cell extract having an enhanced activity of an inhibitor of an enzyme or other cellular component. The extract can be from a cell having at least one inhibitor of at least one enzyme that catalyzes hydrolysis of high-energy phosphate bonds; or the extract can be from a cell having at least one inhibitor of at least one enzyme that catalyzes hydrolysis or formation of phosphodiester bonds, or the extract can be from a cell having at least one inhibitor of at least one enzyme that catalyzes hydrolysis of peptide bonds.

In a further aspect, the in vitro protein or biological macromolecules synthesis system can include one or more nucleic acid templates, and one or more components selected from, at least one inhibitor of an enzyme that degrades the template, at least one extract from a cell having reduced degradative effect on the template, or at least one enzyme with reduced activity to catalyze hydrolysis or formation of phosphodiester bonds. The in vitro protein or biological macromolecules synthesis system can further include addition of an exogenous inhibitor of phosphatases, proteases nucleases, deoxyribonucleases, or ribonucleases.

In a further aspect, the in vitro protein or biological macromolecule synthesis system can further include a molecular chaperone or a foldase. The molecular chaperone or foldase includes, but is not limited to, GroEL/ES, GroEL, GroES, TF, DnaK, DnaJ, GrpE, ClpB, FkpA, Skp, DsbA, DsbC, peptidyl prolyl cis/trans isomerase (PPI), chaperonin 60, chaperonin 10, TCP1, TF55, heat shock protein 60, Cpn60, heat shock protein 10, Cpn10, Lim protein, or signal recognition particle. In a detailed aspect, the in vitro protein or biological macromolecules synthesis system includes a combination of chaperones or foldases in the reaction mixture including, but not limited to, GroEL/ES, TF, DnaK, DnaJ, GrpE, ClpB, FkpA, Skp, DsbA and DsbC.

In a further embodiment, a composition for in vitro protein or biological macromolecules synthesis includes one or more energy sources providing chemical energy for protein or biological macromolecules synthesis, wherein at least one of the energy sources is a glycolytic intermediate. In a detailed aspect, the glycolytic intermediate is selected from 3-phosphoglycerate, 2-phosphoglycerate 2,3-diphosphoglycerate or 1,3-diphosphoglycerate. In a further detailed aspect, the glycolytic intermediate is 3-phosphoglycerate. In a further aspect, the glycolytic intermediate provides chemical energy for protein or biological macromolecules synthesis without addition of exogenous enzymatic cofactor to the composition. In a detailed aspect, the exogenous enzymatic cofactor includes, but is not limited to, thiamine pyrophosphate, FAD⁺, FADH, NAD⁺, NADH, NADP⁺, or NADPH. In a further aspect, the exogenous enzymatic cofactor is NAD⁺ or NADH. In a further aspect, the composition includes a nucleic acid template selected from a DNA template or an RNA template, or the composition includes a cell extract. In a further aspect, the composition for in vitro protein or biological macromolecule synthesis excludes the addition of an exogenous enzyme selected, for example, from pyruvate kinase, acetate kinase, pyruvate oxidase, creatine kinase, or creatine phosphokinase.

Some such compositions for in vitro protein or biological macromolecules synthesis further include, but are not limited to, one or more components selected from at least one extract from a cell having reduced activity of at least one enzyme that catalyzes hydrolysis of high-energy phosphate bonds or hydrolysis or formation of phosphodiester bonds, or at least one inhibitor of at least one enzyme that catalyzes hydrolysis of high-energy phosphate bonds or hydrolysis or formation of phosphodiester bonds, or at least one inhibitor of an enzyme that degrades the template, or at least one extract from a cell having reduced degradative effect on the template. In a further aspect the composition for in vitro protein or biological macromolecules synthesis further includes an inhibitor of a phosphatase or a protease. In a detailed aspect, the composition further includes adenosine triphosphate. In a detailed aspect, the composition further includes cysteine.

A composition for an in vitro protein or biological macromolecule synthesis system is provided. Such compositions can comprise any one or a combination of the elements (e.g., inhibitors, cell extracts, and energy sources) and/or they can also comprise substrates used during transcription and/or translation reactions (e.g., nucleotides, amino acids, polymerases, enzymes, ribosomes, transcription factors, translation factors, cofactors, buffers and buffering salts). In addition, such compositions can comprise any number of products of the transcription and/or translation reaction such as RNA, peptides, or proteins. The compositions can comprise one or more energy sources providing chemical energy for protein or biological macromolecules synthesis, wherein at least one of the energy sources is a glycolytic intermediate, and wherein the glycolytic intermediate provides chemical energy for protein or biological macromolecules synthesis without addition of an exogenous enzymatic cofactor.

In a further embodiment, a kit for in vitro protein or biological macromolecules synthesis includes a nucleic acid template, a cell extract, and one or more energy sources providing chemical energy for protein or biological macromolecules synthesis, wherein at least one of the energy sources is a glycolytic intermediate. In a detailed aspect, the glycolytic intermediate is selected from 3-phosphoglycerate, 2-phosphoglycerate, 2,3-diphosphoglycerate, or 1,3diphosphoglycerate. In a further aspect, the glycolytic intermediate is 3-phosphoglycerate. In a further aspect, the glycolytic intermediate provides chemical energy for protein or biological macromolecule synthesis without addition of an exogenous enzymatic cofactor to the kit. In a detailed aspect, the exogenous enzymatic cofactor includes, but is not limited to, thiamine pyrophosphate, FAD⁺, FADH, NAD⁺, NADH, NADP⁺, NADPH. In a further aspect, the exogenous enzymatic cofactor is NAD⁺ or NADH. In a detailed aspect, the kit excludes the addition of an exogenous enzyme selected, for example, from pyruvate kinase, acetate kinase, pyruvate oxidase, creatine kinase, or creatine phosphokinase.

Some such kits for in vitro synthesis can contain any number or combination of reagents or components. Kits described herein comprise one or more elements selected from the group consisting of one or more components (e.g., energy sources, cell extracts, inhibitors) one or more nucleotides or derivatives thereof, one or more amino acids or derivatives thereof, one or more polymerases, one or more enzymes, one or more cofactors, one or more buffers or buffer salts, one or more energy sources, one or more cell extracts (the cell extracts providing components selected from enzymes, ribosomes, transcription factors, translation factors or co-factors), one or more nucleic acid templates, one or more reagents to determine the efficiency of the kit or assay for production of the products such as nucleic acid and protein products, and directions or protocols for carrying out the methods or to use of the kits and/or its components. The kits can comprise one or more of the above components in any number of separate containers, tubes, vials and the like or such components can be combined in various combinations in such containers.

In further embodiment, a method for producing protein or biological macromolecules from a nucleic acid template in an in vitro protein or biological macromolecules synthesis system includes contacting the nucleic acid template with one or more energy sources providing chemical energy for protein or biological macromolecules synthesis to form a mixture, wherein at least one of the energy sources is a glycolytic intermediate. In a detailed aspect, the glycolytic intermediate is selected from 3-phosphoglycerate, 2-phosphoglycerate, 2,3-diphosphoglycerate, or 1,3-diphosphoglycerate. In a further aspect, the glycolytic intermediate is 3-phosphoglycerate. In a further aspect, the glycolytic intermediate provides chemical energy for protein or biological macromolecule synthesis without addition of an exogenous enzymatic cofactor. In a detailed aspect, the exogenous enzymatic cofactor includes, but is not limited to, thiamine pyrophosphate, FAD⁺, FADH, NAD⁺, NADH, NADP⁺, NADPH. In a further aspect, the exogenous enzymatic cofactor is NAD⁺ or NADH. In a detailed aspect, the method for producing protein or biological macromolecules from a nucleic acid template in an in vitro protein or biological macromolecules synthesis system excludes the addition of an exogenous enzyme selected from pyruvate kinase, acetate kinase, pyruvate oxidase, creatine kinase, or creatine phosphokinase. The method further includes contacting the nucleic acid template and the one or more energy sources with a cell extract, and incubating the mixture under conditions sufficient to produce at least one polypeptide or one nucleic acid encoded by the template.

In further embodiment, a method for constructing an in vitro protein or biological macromolecules synthesis system includes contacting a nucleic acid template with at least one cell extract, contacting one or more energy sources providing chemical energy for protein or biological macromolecules synthesis with the nucleic acid template and the at least one cell extract. The method further provides at least one of the energy sources is a glycolytic intermediate. In a detailed aspect, the glycolytic intermediate is selected from 3-phosphoglycerate, 2-phosphoglycerate, 2,3-diphosphoglycerate, or 1,3-diphosphoglycerate. In a further aspect, the glycolytic intermediate is 3-phosphoglycerate. In a further aspect, the glycolytic intermediate provides chemical energy for protein or biological macromolecule synthesis without addition of an exogenous enzymatic cofactor. In a detailed aspect, the exogenous enzymatic cofactor is NAD⁺.

Improved methods for in vitro synthesis provide for the production of various products in vitro including RNA or other nucleic acid molecules, and peptides or proteins. In one aspect, RNA or other nucleic acid molecules can be produced by mixing one or more nucleic acid templates and at least one component (e.g., one or more energy sources providing chemical energy for protein or biological macromolecule synthesis, wherein at least one of the energy sources is a glycolytic intermediate, and wherein the glycolytic intermediate provides chemical energy for protein or biological macromolecule synthesis without addition of an exogenous enzymatic cofactor, and incubating the mixture under conditions sufficient to produce one or more nucleic acid molecules (e.g., RNA) complementary to all or a portion of the template. In another aspect, peptides or proteins can be produced by mixing one or more nucleic acid templates (e.g., DNA or RNA) and at least one component (an energy source which is a glycolytic intermediate, for example, 3-phosphoglycerate), in the absence of an exogenous enzymatic cofactor, for example, NAD⁺, and incubating the mixture under conditions sufficient to produce one or more peptides or proteins encoded by all or a portion of the template. The methods can further comprise additional steps for further processing the products produced. For example, the produced nucleic acid molecules and proteins can be used to produce other products. They can be used in activity or functional assays or they can be further purified. As will be appreciated, the methods can also be carried out in the presence of one or more other components such as one or more nucleotides or derivatives thereof (which can be detectably labeled), one or more amino acids or derivatives thereof, one or more polymerases, ribosomes, transcription factors, translation factors, one or more cofactors, one or more buffers and/or buffering salts, one or more energy sources, one or more cell extracts, one or more nucleic acid templates and the like. A method for producing protein or biological macromolecules from a nucleic acid template in an in vitro system comprises contacting the nucleic acid template with one or more energy sources providing chemical energy for protein or biological macromolecules synthesis, wherein at least one of the energy sources is a glycolytic intermediate, for example, 3-phosphoglycerate, and wherein the glycolytic intermediate provides chemical energy for protein or biological macromolecule synthesis without addition of an exogenous enzymatic cofactor to form a mixture; and incubating the mixture under conditions sufficient to produce at least one protein encoded by the template. In a detailed aspect of the method, the exogenous enzymatic cofactor includes, but is not limited to, thiamine pyrophosphate, FAD⁺, FADH, NAD⁺, NADH, NADP⁺, or NADPH. In a detailed aspect, the method excludes an exogenous enzyme selected from pyruvate kinase, acetate kinase, pyruvate oxidase, creatine kinase, or creatine phosphokinase. If desired, any detection method known in the art can be used to monitor product (e.g., RNA and protein) production. Inhibition of an enzyme can be accomplished by selected growth conditions in place of or in addition to adding an energy source.

More specifically, methods for producing protein or biological macromolecules from a nucleic acid template in an in vitro synthesis system further provide combining the cell extract and the one or more energy sources with at least one or at least two components selected from the group consisting of at least one inhibitor of at least one enzyme, e.g., an enzyme selected from the group consisting of a nuclease, a phosphatase, a protease, and a polymerase; and at least one cell or extract for synthesis, wherein the cell or extract is modified from a native or wild type extract to exhibit reduced activity of at least one enzyme, e.g., an enzyme selected from the group consisting of a nuclease, a phosphatase, a protease and a polymerase.

### BRIEF DESCRIPTION OF THE DRAWINGS

FIG. 1: Glycolytic reaction demonstrating ATP regeneration utilizing phosphoenolpyruvate and pyruvate kinase.

FIG. 2: Glycolytic reaction demonstrating ATP regeneration utilizing 3-phosphoglycerate, 2-phosphoglycerate, 2,3-diphosphoglycerate or 1,3-diphosphoglycerate.

FIG. 3A: Real time expression of green fluorescent protein (GFP) utilizing two different energy sources in an in vitro transcription translation (IVTT) system.

FIG. 3B: Real time expression and yield of green fluorescent protein (GFP) utilizing two different energy sources in an IVTT system.

FIG. 4: Expression of HIV-RT p66 subunit in a IVTT system.

FIG. 5: Effect of polar lipids on expression of green fluorescent protein (GFP) in an IVTT system.

FIG. 6A: Expression of His-tagged K-ras in the presence and absence of chaperones.

FIG. 6B: Expression of GADD 151 in the presence or absence of chaperones.

FIG. 7: Expression of CAT and GFP using plasmid and PCR products.

FIG. 8: Expression of CAT as linear template in BL21 in the presence of Gam activity from E. coli 12.

FIG. 9: Incorporation of fluoro-tyrosine into GFP and CAT.

FIG. 10: Expression of GFP utilizing 3-PGA and citrate as energy sources.

### DETAILED DESCRIPTION

In vitro protein or biological macromolecules synthesis systems, methods and kits embody one or more of features herein. The in vitro protein or biological macromolecules synthesis system includes the necessary components to synthesize nucleic acids from nucleic acid templates and proteins from nucleic acid templates. The in vitro protein or biological macromolecules synthesis system provides efficient synthesis outside the confines of a cell. The methods are useful for making systems or compositions and for using the systems to produce product molecules of interest. The kits allow or facilitate the practice of the in vitro protein or biological macromolecules synthesis systems.

The in vitro system includes a nucleic acid template that encodes a desired nucleic acid (e.g., RNA or mRNA) and/or protein. The nucleic acid template can be any template, e.g., DNA, RNA, especially mRNA, and can be in any form (e.g., linear, circular, supercoiled, single stranded, double stranded). Such templates are selected for their ability to guide production of the desired protein or biological macromolecule. The desired protein can be any polymer of amino acids encoded by a nucleic acid template to produce a polypeptide molecule. The protein can be further processed coincident with or after synthesis. When desired, the system can be altered as known in the art such that codons will encode for a different amino acid than is normal, including unconventional or unnatural amino acids (including detectably labeled amino acids).

The in vitro protein or biological macromolecules synthesis systems, methods and kits provide a number of advantages. For example, (1) Directed synthesis of active proteins provides high yields; (2) Expression of gene(s) can be accomplished within a day by using PCR or plasmid DNA templates; (3) Proteins which are toxic in living cells can be synthesized in vitro; (4) In vitro evolution of proteins can be done by ribosome and mRNA display procedures; (5) Uniform and specific incorporation of stable isotopes (¹³C, ¹⁵N) for NMR spectroscopy can be accomplished; (6) Directed incorporation of un-natural or modified amino acids can be used to study protein function and to create new proteins with new biological activities; (7) The in vitro system, being an open system, can be modified to any requirements—incorporating, for example, co-factors, chaperones, foldases, phosphatase inhibitors or protease inhibitors; (8) Complexes of several proteins can be synthesized; and (9) Parallel (high throughput) synthesis of proteins can be accomplished by the in vitro protein synthesis system.

The in vitro protein or biological macromolecules synthesis systems, methods and kits solve a number of limitations of cell free transcription translation (CFTT) system. A CFTT system requires a constant supply of energy in the form of ATP/GTP (4 ATP/GTP per peptide bond). Problems with the CFTT system can lead to unproductive depletion of the energy source.

The in vitro protein or biological macromolecules synthesis system provides a stable energy source, for example, a glycolytic intermediate. The glycolytic intermediate is selected from 3-phosphoglycerate, 2-phosphoglycerate, 2,3-diphosphoglycerate, or 1,3-diphosphoglycerate. The energy source can be supplied by dialysis or constant feed. DNA template (supercoil or PCR) stability and mRNA stability is maintained by utilization of cell extracts from nuclease deficient strains, for example, deficient in RNase I or RNaseE. Synthesis of inhibitory byproduct, for example, inorganic phosphate, can be prevented or minimized by utilization of dialysis or constant feed, or utilization of a stable energy source, for example, a glycolytic intermediate selected from 3-phosphoglycerate, 2-phosphoglycerate, 2,3-diphosphoglycerate, or 1,3-diphosphoglycerate.

In this application, unless otherwise indicated, terms have their usual meaning in the context of the art of protein or biological macromolecules synthesis from genetic coding material. For example:

Transcription is the synthesis of a ribonucleic acid (RNA) molecule, e.g., messenger RNA (mRNA), ribosomal RNA, transfer RNA, from a deoxyribonucleic acid (DNA) template.

Translation is the synthesis of a polypeptide, e.g., a protein, from an mRNA template.

Deoxyribonucleic acid (DNA) is a nucleic acid that is a common molecular basis of heredity. Common forms of DNA are single stranded and double stranded. Double stranded DNA is constructed of a double helix held together by hydrogen bonds between purine (adenine or guanine) and pyrimidine (cytosine or thymine) bases which project inward from two chains containing alternate links of deoxyribose and phosphate. An embodiment contemplates using any form of DNA, including cDNA, recombinant DNA, isolated DNA and synthetic DNA.

Ribonucleic acid (RNA) is a nucleic acid that contains ribose and all four major bases (adenine, guanine, cytosine, or uracil) as found in DNA, except uracil is present instead of thymine. RNAs are associated with the control of cellular chemical activities. Messenger RNA (mRNA) is an RNA that serves as a template for protein synthesis. RNA that encodes the protein of interest can be added to the system or can be produced from DNA present or added to the system.

“Nucleotide” refers to a base-sugar-phosphate combination. Nucleotides are monomeric units of a nucleic acids such as DNA and RNA. Nucleotides are incorporated into nucleic acids by DNA or RNA polymerases. The term nucleotide includes, for example, ribonucleoside triphosphates such as ATP, CTP, ITP, UTP GTP, TTP or derivatives thereof and deoxyribonucleoside triphosphates such as dATP, dCTP, dITP, dUTP, dGTP, dTTP or derivatives thereof. Such derivatives include any monomer that can be incorporated into a polynucleic acid molecule, for example, [³⁵S]dATP, 7-deaza-dGTP and 7-deaza-dATP. The term “nucleotide” also refers to dideoxyribonucleoside triphosphates (ddNTPs) and their derivatives. Illustrative examples of dideoxyribonucleoside triphosphates include, but are not limited to, ddATP, ddCTP, ddGTP, ddITP, and ddTTP. A “nucleotide” can be unlabeled or can be detectably labeled by well known techniques. Detectable labels include, for example, different isotopes such as radioactive isotopes, fluorescent labels, chemiluminescent labels, bioluminescent labels and enzyme labels.

The phrase, “in vitro”, refers to systems outside a cell or organism and can be referred to as in vitro transcription translation (IVTT) or as a cell free transcription translation system (CFTT) system. In vivo systems relate to essentially intact cells whether in suspension or attached to or in contact with other cells or a solid. In vitro systems have an advantage of being more manipulatable. Delivering components to a cell interior is not a concern; and manipulations incompatible with continued cell function are also possible, in an in vitro system. However, in vitro systems involve disrupted cells or the use of various components to provide the desired function and thus spatial relationships of the cell are lost. When an in vitro system is prepared, components, possibly critical to the desired activity can be lost with discarded cell debris. Thus in vitro systems are more manipulatable and can function differently from in vivo systems.

The phrase, “in vitro synthesis” or “in vitro protein or biological macromolecules synthesis” or “in vitro protein synthesis” or “cell-free transcription translation (CFTT) system” refers to the cell-free synthesis of polypeptides in a reaction mixture comprising biological extracts and/or defined reagents. The reaction mix will comprise ATP as an energy source and/or an energy source in an energy regenerating system; a template and components for production of the macromolecule, e.g., DNA, mRNA, amino acids, nucleotides and such co-factors, enzymes and other reagents that are necessary for the synthesis, e.g., ribosomes, transfer RNA (tRNA), polymerases, and transcriptional factors. The cell free synthesis reaction can be performed as batch, continuous flow, or semi-continuous flow.

A “reaction mix” or “reaction mixture” refers to a mixture of reaction components, optionally including a cell free extract and exogenously-added components, capable of catalyzing the synthesis of polypeptides from a nucleic acid template. The mixture can comprise metabolic inhibitors that decrease undesirable enzymatic reactions. Alternatively, or in combination, the enhanced reaction mix will be engineered through genetic or other processes to decrease the enzymatic activity responsible for undesirable side-reactions, that result in amino acid depletion or accumulation.

In one embodiment, the reaction mixture comprises extracts from bacterial cells, e.g., E. coli S30 extracts, as is known in the art. For convenience, the organism used as a source of extracts can be referred to as the source organism. While such extracts are a useful source of ribosomes and other factors necessary for protein synthesis, they can also contain small amounts of endogenous enzymes responsible for undesirable side-reactions that are unrelated to protein synthesis, but which deplete ATP, pyruvate or other reagents.

A “nucleic acid template” is a polynucleic acid that serves to direct synthesis of another nucleic acid template or synthesis of a protein. The template is a molecule composed of numerous nucleotide subunits, but can vary in length and in the type of nucleotide subunits. DNA and RNA, e.g., mRNA, are species of nucleic acids that can be used as templates for protein and nucleic acid synthesis. A DNA template is transcribed to form an RNA template complementary to all or a portion of the template. An RNA template is translated to produce a protein or peptide encoded by all or a portion of the template. Thus, the template in a synthesis reaction is one or more species of nucleic acid that codes directly or indirectly for desired protein(s).

A “protein” is a molecule of polymerized amino acids (or derivatives thereof) covalently bonded to one another through peptide bonds. In the present context, a polypeptide is a class of proteins. Proteins produced in accordance with the present can be assayed by any method known in the art. For example, assays specific to the produced protein or more general assays, such as radioactive assays can be used, e.g., using incorporation of ³⁵S-Met into the polypeptide chain.

To provide energy for the in vitro protein or biological macromolecules synthesis reaction, the system includes added energy sources in an “energy regenerating system” to regenerate high-energy phosphate or triphosphate compounds. Energy sources include, but are not limited to glucose, pyruvate, phosphoenolpyruvate (PEP), carbamoyl phosphate, acetyl phosphate, creatine phosphate, phosphopyruvate, 3-phosphoglycerate, 2-phosphoglycerate, triose phosphate, glyceraldehyde-3-phosphate, fructose-1,6-diphosphate, fructose-6-phosphate, glucose-1-phosphate, glucose-6-phosphate, and glucose, that can generate or regenerate high-energy triphosphate compounds. The high-energy triphosphate compounds include, but are not limited to ATP, GTP, UTP, and CTP. One or more of such energy sources can be used and in any combination or order of addition.

In one embodiment, the in vitro protein or biological macromolecules synthesis system comprising one or more energy sources providing chemical energy for protein or biological macromolecule synthesis, wherein at least one of the energy sources is a glycolytic intermediate, and wherein the glycolytic intermediate provides chemical energy for protein or biological macromolecule synthesis without addition of an exogenous enzymatic cofactor. In a further detailed embodiment, one or more energy sources includes the glycolytic intermediate, 3-phosphoglycerate. In a further embodiment the glycolytic intermediate includes, but is not limited to, 3-phosphoglycerate, 2-phosphoglycerate, triose phosphate, 2,3-diphosphoglycerate, 1,3-diphosphoglycerate, glyceraldehyde-3-phosphate, dihydroxyacetone phosphate, fructose-1,6-diphosphate, fructose-6-phosphate, glucose-1-phosphate, glucose-6-phosphate, and glucose. In one aspect, the glycolytic intermediate is present in the reaction mixture at an initial concentration of at least about 1 mM. In a further aspect, the glycolytic intermediate is present in the reaction mixture at an initial concentration of at least about 10 mM. In a further embodiment, the glycolytic intermediate provides chemical energy for protein or biological macromolecule synthesis without addition of an exogenous enzymatic cofactor, NAD⁺ or NADH. For example, when 3-phosphoglycerate is used as an added energy source to the IVTT system, the amount of protein synthesized can be 2 fold or greater compared to when PEP is added as an energy source to the IVTT system. As a further example, when 3-phosphoglycerate is used as an added energy source to the IVTT system, the amount of protein synthesized can be 5 fold or greater, 10 fold or greater, 15-fold or greater, or 20-fold or greater compared to when PEP is added as an energy source to the IVTT system.

The term “endogenous” is used to refer to enzymes, factors, cofactors and other compounds present in the cell extract. “Exogenous” components are those that are introduced into the cell extracts through addition, and can be added at the time of synthesis, or can be added through genetic or other manipulation to the cells used as the starting material for extracts. For example, plasmids encoding an exogenous enzyme of interest can be incorporated into the bacterial cells prior to preparation of the extracts. For example, an exogenous enzymatic cofactor of interest can be added to the reaction mixture at the time of in vitro protein or biological macromolecule synthesis. In one embodiment, exogenous enzymatic cofactors are excluded from the IVTT system. In a further embodiment, exogenous enzymes are excluded from the IVTT system

The one or more energy sources include a glycolytic intermediate for energy regeneration without addition of an exogenous enzymatic cofactor to the IVTT system. Examples of an exogenous enzymatic cofactor include, but are not limited to, thiamine pyrophosphate, flavin adenine dinucleotide (FAD⁺), reduced flavin adenine dinucleotide (FADH), flavin mononucleotide (FMN), nicotinamide adenine dinucleotide (NAD⁺), reduced nicotinamide adenine dinucleotide (NADH), nicotinamide adenine dinucleotide phosphate (NADP⁺), or reduced nicotinamide adenine dinucleotide phosphate (NADPH). Flavin nucleotides, for example, FAD or FMN, are coenzymes of the flavin enzymes. Flavin enzymes are a diverse group of 70 or more oxidoreductases of animals, plants and microorganisms. Flavin enzymes can be oxidases, reductases, or dehydrogenases. Nicotinamide adenine dinucleotide (NAD) is a pyridine nucleotide coenzyme involved in many biochemical redox processes. It is the coenzyme of a large number of oxidoreductases, which are classified as pyridine nucleotide-dependent dehydrogenases. Mechanistically, it serves as the electron acceptor in the enzymatic removal of hydrogen atoms from specific substrates.

The one or more energy sources include a glycolytic intermediate for energy regeneration without addition of an exogenous enzyme to the IVTT system. An exogenous energy source, for example, 3-phosphoglycerate (3-PGA), can be added as an energy regenerating system. Examples of an exogenous enzyme include, but are not limited to, pyruvate kinase which can be used in combination with PEP, acetate kinase which can be used in combination with acetyl phosphate, pyruvate oxidase which can be used in combination with pyruvate, creatine kinase which can be used in combination with creatine phosphate, creatine phosphokinase which can be used in combination with creatine phosphate.

In the case where sufficient energy is not initially present in the synthesis system, an additional source of energy is supplemented. The supplement can be delivered continuously or can be delivered in one or more discreet supplements. In one embodiment includes addition of at least one energy source (or two or more, or three or more, four or more, five or more, six or more energy sources.) to provide the energy for the synthesis reactions. Often the energy source(s), especially added energy source(s), can contain one, two, or three high-energy phosphate bonds. Often the energy source(s), especially added energy source(s), can contain three high-energy phosphate bonds.

The two or more energy sources can include, for example, a glycolytic intermediate and a tricarboxylic acid (TCA) cycle intermediate in combination with an exogenous enzymatic cofactor for energy regeneration and without addition of an exogenous enzyme to the IVTT system. In a further embodiment, the TCA cycle intermediate includes, but is not limited to, citrate, isocitrate, malate, oxaloacetate, α-ketoglutarate, succinate or fumarate. Two exogenous energy sources, for example, 3-phosphoglycerate (3-PGA) and citrate in combination with NAD⁺, can be added as an energy regenerating system.

The energy source can be present in any amount that is suitable for the desired synthesis. For example, the chemical energy source can be added to achieve a concentration of from 5-100 mM. About 10, 15, 20, 25, 30, 50, 60, 70, 80 or 90 mM can also be target concentrations. The precise concentration will vary as synthesis consumes energy and the energy is replenished from these sources. The concentration can be controlled within various ranges, for example about 5-100 mM, 10-90 mM, 20-80 mM, or 30-60 mM. Any target concentration can be used as an approximate boundary for the desired range of concentration of energy source. Energy sources can also be added or supplemented during the in vitro synthesis reaction.

When two or more energy sources are used, each source can independently be targeted to be one of these target concentrations. When multiple energy sources are included in the system, synthesis (especially protein synthesis) is found to be accelerated and prolonged in time, so that protein and/or nucleic acid products are more efficiently produced by the synthesis system. For example, the two or more energy sources added to the in vitro transcription translation system can include, but are not limited to, glucose, pyruvate, phosphoenolpyruvate (PEP), carbamoyl phosphate, acetyl phosphate, creatine phosphate, phosphopyruvate, 3-phosphoglycerate, 2-phosphoglycerate, triose phosphate, glyceraldehyde-3-phosphate, fructose-1,6-diphosphate, fructose-6-phosphate, glucose-1-phosphate, glucose-6-phosphate, and glucose. For example, when phosphoenol pyruvate (PEP) and acetyl phosphate are used as added energy sources, the amount of protein synthesized can be more than doubled as compared to when only acetyl phosphate is added. Thus, an embodiment includes an in vitro protein or biological macromolecule synthesis system that comprises at least one, or at least two or more different energy sources that provide high-energy phosphate bonds for the synthesis reactions.

For example, the two or more energy sources added to the in vitro transcription translation system can further include, but are not limited to, TCA cycle intermediates, for example, malate, oxaloacetate, citrate, isocitrate, α-ketoglutarate succinate, or fumarate.

Compositions and methods are provided to enhance the in vitro protein or biological macromolecule synthesis includes one or more energy sources in combination with a cell extract providing chemical energy for protein or biological macromolecule synthesis. In one aspect, at least one energy source is a glycolytic intermediate. The glycolytic intermediate provides chemical energy without addition of an enzyme or enzymatic cofactor that is exogenous to the cell extract that is included in the in vitro protein or biological macromolecule synthesis system. In a further aspect, at least two energy sources are a glycolytic intermediate and a TCA cycle intermediate. The glycolytic intermediate and the TCA cycle intermediate in combination with an exogenous enzymatic cofactor provide chemical energy without addition of an enzyme exogenous to the cell extract that is included in the in vitro protein or biological macromolecule synthesis system. The one or more energy sources generate or regenerate high-energy triphosphate compounds. In one embodiment, the high-energy triphosphate compound is adenosine triphosphate (ATP). In a further embodiment, the high-energy triphosphate compound is guanosine triphosphate (GTP), cytosine triphosphate (CTP), and/or thymidine triphosphate (TTP).

In a further aspect, the in vitro protein or biological macromolecules synthesis system further comprises exogenous adenosine triphosphate (ATP) and/or cysteine added to the IVTT system to replenish depleted endogenous amounts of ATP and/or cysteine.

An “inhibitor of at least one enzyme that catalyzes hydrolysis of high-energy phosphate bonds” is an inhibitor that reduces or eliminates the enzymatic activity of a phosphatase, for example, an inhibitor of E. coli alkaline phosphatase.

An “inhibitor of at least one enzyme that catalyzes hydrolysis of peptide bonds” is an inhibitor that reduces or eliminates the enzymatic activity of a protease, for example, an inhibitor of an E. coli protease.

A “protease inhibitor” is a compound added to a reaction mixture that reduces or eliminates protease enzyme activity and reduces or eliminates cleavage of peptide bonds in a polypeptide produce of the cell free transcription translation system.

A “phosphatase inhibitor” is a compound added to a reaction mixture that reduces or eliminates phosphatase enzyme activity and reduces or eliminates cleavage of phosphate from a high-energy phosphate compound, for example, ATP, GTP, CTP, UTP, 3-phosphoglycerate, 2-phosphoglycerate, or phosphoenolpyruvate, in the cell free transcription translation system.

An “inhibitor of at least one enzyme that catalyzes hydrolysis or formation of phosphodiester bonds” is an inhibitor of a nuclease, for example, a deoxyribonuclease, a ribonuclease, a helicase, or an E. coli helicase

An “inhibitor of an enzyme that degrades a nucleic acid template” is an inhibitor that reduces or eliminates the enzymatic activity of a nuclease, for example, a deoxyribonuclease, a ribonuclease or a helicase.

A nuclease is a compound with enzymatic activity resulting in the hydrolysis of polynucleotides or polynucleic acids. Deoxyribonuclease (DNase) cleaves DNA. Ribonuclease (RNase) cleaves RNA. As a rule, the phosphodiester bonds of both DNA and RNA are resistant to hydrolysis. Biological systems have compensated by producing many nucleases that have the ability to accelerate hydrolysis of these bonds. There are various ways of classifying nucleases. For example, an endonuclease is a nuclease that cleaves its nucleic acid substrate at internal sites in the nucleotide sequence. An exonuclease is a nuclease that cleaves nucleotides sequentially from free ends of its nucleic acid substrate. Some nucleases have both endonuclease and exonuclease activity.

Nucleases have various functions relating to their specificities, for example, DNA repair activity, 3′ to 5′ or 5′ to 3′ specificity, double stranded or single stranded DNA or RNA:DNA specificity. Nucleases can produce a 3′-terminal a,p-unsaturated aldehyde and 5′ terminal phosphate (AP lyases); a 3′-hydroxyl nucleotide and 5′-terminal phosphate (class II endonuclease); or a 5′-hydroxyl and 3′-phosphate (class IH endonuclease). Nuclease can process one or more types of polynucleotide, for example, single stranded DNA, double stranded DNA, mRNA, oligoRNA, ribosomal RNA. Restriction nucleases are nucleases that cleave at sites with specific nucleotide sequences.

Additional properties and types of nucleases are known in the art as described in the many publications relating to nucleases that are available. Nucleases, Second Edition, 1993, Linn et al, eds. Cold Spring Harbor Laboratory Press is specifically referred to and hereby incorporated in its entirety by reference for all purposes. One aspect relates to removing, preventing or inhibiting any one or more of the nuclease functions or to using cell extracts from cells having one or more nuclease or other activity deleterious or damaging to the nucleic acid template or other substrate reduced, substantially reduced or eliminated by, for example, modifying or mutating one or more genes encoding such activities.

An “extract” or “cell extract” is a cell lysate or exudate. The cell can be any cell that can be grown for preparing an extract. Both prokaryotic cells and eukaryotic cells can be used for protein and/or biological macromolecules synthesis. Prokaryotic systems benefit from simultaneous or “coupled” transcription and translation. Eukaryotic systems are also used. See e.g., Pelham et al, 1976, European Journal of Biochemistry, 67: 247. The ordinarily skilled artisan will appreciate that different aspects will be more or less advantageous depending on the type of cell or system, for example eukaryotic or prokaryotic systems, or the specific cell or cell line. The cell extract can be an E. coli S30 cell extract. Methods for producing active extracts can be found, for example, in Pratt, 1984, Coupled Transcription-Translation In Prokaryotic Cell-Free Systems, 179-209, and in Hames, B. D. and Higgins, S. J. (ed.), Transcription and Translation: a practical approach, IRL Press, New York. One can modify the E. coli cell-free S30 extract by collecting the ribosome fraction from the S30 by ultracentrifugation. See e.g., Kudlicki et al., 1992, Anal Biochem 206: 389-93.

The cell extract can be from an E. coli strain that express genes for rare t-RNAs not normally found or found in low quantities in E. coli, selected from tRNAs for amino acids arginine (AGG, AGA), proline (CCC), glycine (GGA), leucine (CUA), or isoleucine (AUA). These rare E. coli tRNAs are useful for expression of eukaryotic proteins in a cell free transcription translation system in E. coli. For example, the Rosetta™ host strains (Novagen, Madison, Wis.) enhance the expression of eukaryotic proteins that contain codons rarely used in E. coli. These strains supply tRNAs for AGG, AGA, AUA, CUA, CCC, GGA codons on a compatible chloramphenicol-resistant plasmid. Thus the Rosetta strains provide for “universal” translation which is otherwise limited by the codon usage of E. coli. The tRNA genes are driven by their native promoters. In Rosetta™ (DE3)pLysS and Rosetta™ (DE3)pLacI, the rare tRNA genes are present on the same plasmids that carry the T7 lysozyme and lac repressor genes, respectively.

The in vitro protein or biological macromolecule synthesis system can include a cell extract to which components selected from lipids, cholesterol, or membranes are added to the cell extract. During preparation of E. coli extract for cell-free protein synthesis, the membranes are generally removed during centrifugation. In some cases, particularly during synthesis of membrane-associated proteins in the cell-free system, it is advantageous to add membranes, cholesterol, or lipids as anchors. For cell free protein synthesis of membrane proteins, e.g., G-protein coupled receptors (GPCR), E. coli polar lipids (Avanti® Polar Lipids, Inc., Alabaster, Ala.) are added to the IVTT system and improve the yield of the membrane protein in the IVTT system.

Compositions and methods are provided to enhance the synthesis by addition of cell extracts. In one aspect the in vitro protein or biological macromolecule synthesis system includes a cell extract to provide components for in vitro transcription and translation selected from enzymes, ribosomes, transcription factors, translation factors or co-factors. In a further aspect, the cell providing the extract or components for synthesis is modified or mutated to inhibit or inactivate unwanted components/proteins/enzymes in the synthesis reaction. For example, mutations can be made in RNases, such as RNase E, or in other enzymes, such as alkaline phosphatase, protease, and endonuclease A. In addition, inhibitors, such as inhibitors of nucleases that act on nucleic acid templates (e.g., Gam protein of phage lambda to inhibit RecBCD) or inhibitors of other unwanted or detrimental components/proteins/enzymes in the synthesis reaction can be used to enhance the production of desired products in vitro.

Alternatively, or in addition to the modified/mutant cell extracts and/or inhibitors, synthetic pathways of deleterious components/proteins/enzymes can be shut down or slowed, for example, by growth strategies that fail to induce the normal quantities of deleterious components/proteins/enzymes or that fail to provide cofactors for the enzymatic activity, or by inhibitors of transcription or translation of the deleterious genes or proteins. Employing growth strategies that result in diminished enzyme activity is one embodiment for modulating or inhibiting enzymes. The embodiment also relates to these modified/mutated cells and/or genes and media capable of growing cells used in the cell extracts.

Thus, the compositions and methods more generally relates to more efficient synthesis involving, for example, at least one extract from a cell from which at least one enzyme/component/protein that participates in hydrolysis of high-energy phosphate bonds or hydrolysis or formation of phosphodiester bonds is absent, is removed, or is modified to reduce or inactivate its activity (for example, by modulating, mutating, or modifying one or more genes involved in expression or which encode such protein/component/enzyme); at least one inhibitor of at least one enzyme/component/protein that participates in hydrolysis of high-energy phosphate bonds or hydrolysis or formation of phosphodiester bonds; and/or at least two sources of energy to provide chemical energy for the synthesis process. Any one or a number of these features can be combined and used in various in vitro synthesis systems. As will be recognized, the embodiments can also be used to prepare various intermediates of a protein synthesis reaction, if desired. For example, the compositions and methods can be used to prepare RNA (during transcription of a DNA template) and such RNA can be isolated or further processed by standard molecular biology techniques.

In a detailed aspect, the compositions and methods feature embodiments that include an in vitro synthesis system that includes one or more (or combinations thereof) of the following: i) removal, modulation, inhibition or inactivation of at least one activity, e.g., an enzymatic activity, that catalyzes hydrolysis of high-energy phosphate bonds or hydrolysis or formation of phosphodiester bonds or hydrolysis of peptide bonds, for example, an enzyme selected from the group consisting of a nuclease, a phosphatase, a protease and a polymerase; ii) use of at least one cell extract for synthesis, wherein the extract is modified to exhibit reduced activity (compared to an unmodified extract) or inactivation of at least one enzyme that catalyzes hydrolysis of high-energy phosphate bonds or hydrolysis or formation of phosphodiester bonds, for example, an enzyme selected from the group consisting of a nuclease, a, a protease, and a polymerase; and iii) one or more energy sources that supply energy for synthesis. See, for example, U.S. application Ser. No. 60/273,827, filed Mar. 8, 2001 and in PCT International Application WO 02/072890.

The subject system is useful for in vitro protein or biological macromolecules synthesis, which can include the transcription of RNA from DNA or RNA templates. The reactions can utilize a large scale reactor, small scale, or can be multiplexed to perform a plurality of simultaneous syntheses. Continuous reactions will use a feed mechanism to introduce a flow of reagents, and can isolate the end-product as part of the process. Batch systems are also of interest, where additional reagents can be introduced to prolong the period of time for active synthesis. A reactor can be run in any mode such as batch, extended batch, semi-batch, semi-continuous, fed-batch and continuous, and which will be selected in accordance with the application purpose.

Of particular interest is the translation of mRNA to produce proteins, in which translation can be coupled to in vitro synthesis of mRNA from a DNA template. Such a cell-free system will contain all factors required for the translation of mRNA, for example ribosomes, amino acids, tRNAs, supplemented rare tRNAs, aminoacyl-tRNA synthetases, elongation factors and initiation factors. Cell-free systems known in the art include wheat germ extracts (See e.g., Roberts et al., 1973, PNAS USA 70: 2330, reticulocyte extracts (See e.g., Pelham et al., 1976, Eur. J. Biochem. 67: 247) and E. coli extracts, which can be treated with a suitable nuclease to eliminate active endogenous mRNA.

Additional components, for example, cell-free extracts, genetic template, amino acids and energy sources, and other materials specifically required for protein synthesis can be added to the reaction. These materials include, but are not limited to, salt, polymeric compounds, cyclic AMP, inhibitors for protein or nucleic acid degrading enzymes, inhibitors or regulators of protein synthesis, oxidation/reduction adjusters, non-denaturing surfactants, buffer components, spermine or spermidine.

“Chaperones” or “molecular chaperones” are a family of unrelated classes of proteins that mediate the correct assembly of other polypeptides but that are not components of the functional assembled structures. Chaperones are necessary in vivo for cell viability under both normal and stress conditions. Chaperones are also useful to assist proper folding of polypeptides in an in vitro transcription translation system. There are several chaperone systems which carry out a multitude of functions all aimed towards insuring the proper folding of target proteins. Chaperones can assist in the efficient folding of newly-translated proteins as these proteins are being synthesized on the ribosome and can maintain pre-existing proteins in a stable conformation. Chaperones can also promote the disaggregation of preformed protein aggregates. Many of the identified chaperones are also heat shock proteins. The general mechanism by which chaperones carry out their function usually involves multiple rounds of regulated binding and release of an unstable conformer of target polypeptides. The four main chaperone systems in the Escherichia coli cytoplasm are as follows. (1) Ribosome-associated trigger factor that assists in the folding of newly-synthesized nascent chains. (2) The Hsp 70 system consisting of DnaK (Hsp 70), its cofactor DnaJ (Hsp 40), and the nucleotide exchange factor GrpE. This system recognizes polypeptide chains in an extended conformation. (3) The Hsp 60 system, consisting of GroEL (Hsp 60) and its cofactor GroES (Hsp 10), which assists in the folding of compact folding intermediates that expose hydrophobic surfaces. (4) The Clp ATPases which are typically members of the Hsp 100 family of heat shock proteins. These ATPases can unfold proteins and disaggregate preformed protein aggregates to target them for degradation. See, for example, Ellis and Hemmingsen, 1989, Trends Biochem. Sci. 14: 339-342; Houry, 2001, Curr. Protein Pept Sci., 2: 227-244; Schlieker et al. 2002, J. Biotech. 96: 13-21; Hartl and Hayer-Hartl, 2002, Science 295: 1852-1858. Foldases, which accelerate rate-limiting steps along the folding pathway, include: peptidyl prolyl cis/trans isomerases (PPI's) (See e.g., Dartigalongue and Raina, 1998, EMBO J. 17: 3968-3980); Dsb (disulfide bond formation) proteins (See e.g., Wulfing and Pluckthun, 1994, J. Mol. Biol. 242: 655-669; Bardwell J. C. 1994, Mol. Micro. 14: 199-205; Georgiou and Valex, 1996, Curr. Opin. Biotechnol. 7: 190-197); FkpA which catalyze the cis/trans isomerization of prolyl peptide bonds (See e.g., Arie et al. 2001, Mol. Microbiol. 39: 199-210) and Skp (Holck et. Al. 1997, Biochim. Biophys. Acta. 908: 188-199; Hayhurst and Harris, 1999, Prot. Exp. Purif. 15: 336-343). In addition, there are uncharacterized and unknown chaperones and foldases.

The use of chaperones in a cell-free protein synthesis system has been described. See e.g., Kudlicki et. al, 1994, J. Biol. Chem. 269: 16549-16553. In vitro transcription translation synthesis of active rhodanase occurs in the presence of a combination of chaperones. A cell-free protein synthesis system can be made devoid of endogenous chaperones. A chaperone-deficient system was used to study synthesis and folding of bacterial dihydrofolate reductase and of rhodanase, a eukaryotic mitochondrial enzyme. Both enzymes were synthesized in active form and with high specific activity in the chaperone-deficient system as well as in a chaperone proficient host. However, in the chaperone-deficient host, a portion of these two enzymes stayed bound (inactive) to the ribosome. Addition of GroEL and DnaK released the enzymes in an active form. See e.g., Kudlicki et al. 1994, Anal Biochem. 217:12-19.

Different chaperones can work better in vitro or in vivo for some proteins compared to other proteins. For example, the GroEL/ES system can account for folding of no more than 5% of the proteins produced in rapidly growing E. coli. See e.g., Lorimer, 1996, FASEB 10: 5-9. In order to avoid problems of choosing a particular chaperone or a few chaperones, certain embodiments use a cocktail of chaperones and foldases in an in vitro transcription translation system. In an exemplary embodiment, a combination of chaperones useful in an IVTT system includes GroEL/ES, TF, DnaK, DnaJ, GrpE, ClpB, FkpA, Skp and DsbC, leading to improved expression of protein, e.g., His-tagged K-Ras in an IVTT system. Since an IVTT system is an open system, it is possible to incorporate any number of chaperones, co-factors, foldases, protease inhibitors, or other components in the system.

Salts added to the IVTT include, for example, potassium, magnesium, ammonium and manganese salt of acetic acid or sulfuric acid, for example potassium acetate, magnesium acetate, potassium sulfate, or magnesium sulfate. Some of cationic salts can have amino acids as a counter anion. Polymeric compounds added to the IVTT system can be polyethylene glycol, dextran, diethyl aminoethyl, quaternary aminoethyl and aminoethyl. Oxidation/reduction adjusters added to the IVTT system can be dithiothreitol, ascorbic acid, glutathione and/or their oxides. Also, a non-denaturing surfactant such as Triton X-100 can be used at a concentration of 0-0.5 M. Spermine and spermidine can be used for improving protein synthetic ability, and cAMP can be used as a gene expression regulator.

When the concentration of a particular component of the reaction medium is changed, the concentration of other components can be changed accordingly. For example, the concentrations of several components such as nucleotides and energy source compounds can be simultaneously controlled in accordance with the changes in concentration of other components. Also, the concentration levels of components in the reactor can be varied over time.

The reaction is maintained typically in the range of pH 5-10 and a temperature of 20°-50° C., and in the range of pH 6-9 and a temperature of 25°-40° C.

When using a protein isolating means in a continuous operation mode, the product output from the reactor flows through a membrane into the protein isolating means. In a semi-continuous operation mode, the outside or outer surface of the membrane is contacted with predetermined solutions that are changed cyclically in a predetermined order. These solutions contain substrates such as amino acids and nucleotides, for the in vitro transcription/translation system. For this process, the reactor is operated in dialysis, diafiltration batch or fed-batch mode. A feed solution can be supplied to the reactor through the same membrane or through a separate injection unit. Synthesized protein is accumulated in the reactor, and then is isolated and purified according to a known method for protein purification after completion of the system operation.

The direction of flow of liquid reagents can be perpendicular to and/or tangential to a membrane. Tangential flow is effective for recycling ATP and for preventing membrane plugging. Tangential flow can be superimposed on perpendicular flow. Flow perpendicular to the membrane can be effected by a positive pressure pump or a vacuum suction pump. The solution in contact with the outside surface of the membrane can be changed cyclically, and can be in a steady tangential flow with respect to the membrane. The reactor can be stirred internally or externally by proper agitation means.

During protein synthesis in the reactor, the protein-isolating means selectively isolates the desired protein and can include a unit containing antibody coated particles or other particles immobilized with an affinity component for adsorbing the, desired in vitro synthesized protein. The protein isolating means can further comprise a membrane with pores of proper molecular weight exclusion properties for isolating the desired protein at a specific molecular weight. The protein isolating means comprises two columns for alternating use. Alternately, the protein product can be absorbed using expanded bed chromatography, in which case a membrane may or may not be used.

The amount of protein produced in a translation reaction can be measured by various means. One method relies on the availability of an assay which measures the activity of the particular protein being translated. An example of an assay for measuring protein activity is a green fluorescent protein (GFP) assay, a luciferase assay system, or chloramphenicol acetyl transferase (CAT) assay system. These assays measure the amount of functionally active protein produced from the translation reaction. Activity assays will not measure full-length protein that is inactive due to improper protein folding or lack of other posttranslational modifications necessary for protein activity.

Another method to quantitate the amount of protein produced in a coupled in vitro transcription translation reactions is to perform the reaction using a known quantity of radiolabeled amino acid such as ³⁵S-methionine or ³H-leucine and subsequently measuring the amount of radiolabeled amino acid incorporated into the newly synthesized protein. Incorporation assays will measure the amount of radiolabeled amino acids in all proteins produced in an in vitro translation reaction including truncated protein products. The radiolabeled protein can be further separated on a SDS polyacrylamide gel, and by autoradiography confirmed that the in vitro translation product is the proper size and that secondary protein products have not been produced.

An in vitro protein or biological macromolecules synthesis system has many advantages when used in either batch mode, fed batch mode, or semicontinuous systems such as a continuous exchange cell-free system. See e.g., Kigawa et al., 1999, FEBS Lett. 442:15-19. The in vitro protein or biological macromolecules synthesis system can allow the synthesis of proteins toxic to cell division. It also allows most of the metabolic resources to be focused only on product synthesis. More importantly, it provides great flexibility in manipulating protein synthesis and folding. See e.g., Swartz, 2001, Current Opinion in Biotechnology, 12: 195-201.

An in vitro protein or biological macromolecules synthesis system is useful to develop and optimize in vitro display technology. Examples of in vitro display technology include ribosome display and mRNA display. See e.g., Amstutz et al., Current Opinion in Biotechnology, 2001, 12: 400-405. The in vitro protein or biological macromolecules synthesis system improves the quality of the library of polypeptides by increasing the quantity of full length polypeptide chains. In vitro display technologies, such as ribosome display and mRNA display, are valuable tools for applications in addition to selecting compounds that bind to polypeptides. The in vitro display technology has great potential for directed evolution of protein stability and affinity, the generation of high-quality libraries by in vitro preselection, the selection of enzymatic activities, and the display of cDNA and random-peptide libraries. In addition, these technologies have features that make them amenable to standardization and automation: they comprise fast selection cycles, allow the processing of huge libraries, are not limited by cellular transformations, and are not biased by in vivo environments.

An in vitro protein or biological macromolecules synthesis system is useful for in vitro generation of highly specific and fully human antibodies. The in vitro protein or biological macromolecules synthesis system is used to generate the highly variable complementarity determining regions (CDRs) to represent the structural diversity of the human antibody repertoire. The highly variable CDRs are located within the seven heavy chain and seven light chain variable regions of the immunoglobulin genes or Fab fragments. An in vitro mutagenesis procedure allows the synthesis of any desired mixture of amino acids at each single position of the CDRs within the variable regions. In the libraries of polypeptides that are generated, the length and amino acid composition of the CDR regions are retained according to the natural human antibody sequences. Each of the CDR cassettes can be specifically exchanged in order to facilitate the optimization of a specific antibody.

An in vitro protein or biological macromolecules synthesis system is useful to develop and optimize protein microarrays of high affinity and high specificity binding proteins, such as immunoglobulin light chain and heavy chain CDRs, for therapeutic applications. The CDRs are utilized on a protein microarray and assayed for binding to a target molecule, for example, a cytokine, which can be normally difficult to assay by antibody binding due to non-immunogenicity or toxicity of the antigen. The CDRs produced by the in vitro protein synthesis system are useful for detecting target molecules, for example, a cytokine, in a complex biological sample.

Methods for production of protein and/or biological macromolecules molecules in an in vitro synthesis system are included. The methods include producing such products with in vitro systems having at least one, or at least two or three or more energy sources that can be added, either continuously or by one or more discreet additions. The methods also include producing proteins and/or biological macromolecules with in vitro systems modified to maintain the nucleotide template. For example: nucleases, phosphatases, proteases or polymerases can be inhibited by chemical or physical conditions; and/or extracts from cells deficient or mutated or modulated in one or more enzyme, such as nuclease, phosphatase, proteases or polymerase can be used a basis for the system.

Mutated or modified cells can be made as indicated in the examples and/or can be made according to methods known in the art. In the context a modified cell is a cell with one or more mutated or modified genes or modified or modulated in such a way to reduce, substantially reduce, eliminate, inhibit or modulate certain activity or activities of interest. A mutated gene includes a gene that is not transcribed or translated into the fully functional gene product associated with the wild type gene. A mutation can be any mutation that does not result in full function of the wild type gene product. For example, the mutation can be a point mutation, a complete or partial deletion of the gene, a complete or partial substitution of the gene and/or one or more insertions in a gene.

A “cell extract,” as described above, is processed to remove cellular debris. Centrifugation is a common method for removing such solid material. Filtration, chromatography, or any other separation or purification procedure can be used to produce a desired extract. The extract includes all necessary components for synthesis that are not otherwise provided in the system. The extract can be concentrated using one or more of the many tools of the art. Enzymes and other components present in the extract to provide energy and other components for the synthesis reaction can originate in the extracted cell or can be added during the production of the extract. “Cell extract” also includes a mixture of components crafted to imitate a cell lysate or exudate with respect to the components necessary or desired for protein or biological macromolecule synthesis. A cell extract thus can be a mixture of components to imitate or improve upon a cell lysate or exudate in protein synthesis reactions and/or to provide components used for synthesis from a nucleic acid template. Such mixtures, as will be recognized by one of ordinary skill in the art, can be produced by obtaining a partial extract or fraction thereof and/or by mixing any number of individual components. The extracts can be optimized for expression of genes under control of a specific promoter, (for example see Nevin and Pratt, 1991, FEBS Lett 291: 259-63) which system consists of an E. coli crude extract (prepared from cells containing endogenous T7 RNA polymerase) and rifampicin (an E. coli RNA polymerase inhibitor)). Reagent concentrations are optimized to enhance protein production. See e.g., Kim et al., 1996, Eur. J. Biochem. 239: 881-886.

Cell extracts can be made from mutated or modified cells. Such cells can be made as indicated in the examples and/or can be made according to methods known in the art. A modified cell is a cell with one or more mutated or modified genes. The cell can be modified or modulated in such a way as to reduce, substantially reduce, eliminate, inhibit or modulate certain activity or activities of interest. A mutated gene includes a gene that is not transcribed or translated into the fully functional gene product associated with the wild type gene. A mutation can be any mutation that does not result in full function of the wild type gene product. For example, the mutation can be a point mutation, a complete or partial deletion of the gene, a complete or partial substitution of the gene and/or one or more insertions in a gene.

An “extract from a cell having reduced activity of at least one enzyme that catalyzes hydrolysis of high-energy phosphate bonds” can be a cell extract with reduced phosphatase activity in the extract. The cell extract, for example, a bacterial cell, can have a genetic mutation in any one of a number of genes encoding a phosphatase enzyme in the cell resulting in reduced phosphatase activity when used in an in vitro protein or biological macromolecule synthesis system. Alternatively, the cell extract can have phosphatases removed from the extract by protein purification techniques, for example, by affinity chromatography or immune affinity chromatography or the cell extract can be made from cells grown in the presence of a high concentration of phosphate.

An “extract from a cell having reduced activity of at least one enzyme that catalyzes hydrolysis of peptide bonds” can be a cell extract with reduced protease activity in the extract. The cell extract, for example, a bacterial cell, can have a genetic mutation in any one of a number of genes encoding a protease enzyme in the cell resulting in reduced protease activity when used in an in vitro protein or biological macromolecule synthesis system. Alternatively, the cell extract can have proteases removed from the extract by protein purification techniques, for example, by affinity chromatography or immune affinity chromatography.

An “extract from a cell having reduced degradative effect on a nucleic acid template” or an “extract from a cell having reduced activity of at least one enzyme that catalyzes hydrolysis or formation of phosphodiester bonds” can be a cell extract with reduced nuclease activity, e.g., reduced activity of deoxyribonuclease, ribonuclease, or helicase in the extract. The cell extract, for example, a bacterial cell, can have a genetic mutation in any one of a number of genes encoding a nuclease enzyme, for example, a deoxyribonuclease, a ribonuclease, or a helicase, in the cell resulting in reduced nuclease activity when used in an in vitro protein or biological macromolecule synthesis system. Alternatively, the cell extract can have nucleases removed from the extract by protein purification techniques, for example, by affinity chromatography or immune affinity chromatography.

The in vitro protein or biological macromolecule synthesis systems are based on one or more cell extracts added to the in vitro protein or biological macromolecules synthesis system. Cells possess the necessary components to synthesize proteins and/or nucleic acids. Extracts of cells are a readily obtainable source of in vitro synthesis components. Components processed from cells or components processed or synthesized from other sources can be included in the extract. Maintenance of the template is necessary to maximize the duration of the synthesis process. Thus the synthesis system can include components that maintain the template. The template can be maintained by preventing enzymatic, chemical or other degradation of the template.

The in vitro protein or biological macromolecules synthesis system therefore can include modifications to the extract to improve product synthesis. When the extract contains enzymes whose activities compromise protein and/or nucleic acid production, inhibition of these enzymes will result in more efficient synthesis by the system. Thus, in vitro protein or biological macromolecules synthesis systems comprise inhibitors of at least one enzyme. Nuclease, phosphatase, and protease inhibitors are advantageously used to increase protein and/or nucleic acid synthesis efficiency. Inhibition of enzymes that unnecessarily consume compounds used in the synthesis reaction can also improve synthesis efficiency. Depending on the specific enzymes present in the extract, for example, one or more of the many known nuclease, polymerase, protease or phosphatase inhibitors can be selected and advantageously used to improve synthesis efficiency.

However, it may not be desirable to prevent or inhibit the activities of all nucleases. For example, RNase I and RNase I* degrade short RNA oligonucleotides in preference to full length RNAs. In some circumstances this activity can prove helpful. Cells use RNase I and RNase I* to degrade RNA that is no longer needed. For example, mononucleotides made available by RNase I would be available as substrates for transcribing DNA. Alternatively, blocking RNase I can be insufficient by itself to maintain the RNA for translation during the protein synthesis process. For example, other enzymes can be more active in initial degradation of mRNA. See, for example, PCT International Application WO 02/072890.

To maintain the template, cells that are used to produce the extract can be selected for reduction, substantial reduction or elimination of activities of detrimental enzymes or for enzymes with modified activity. Thus, in vitro protein or biological macromolecule synthesis systems comprise extracts of cells having altered activity (for example by modifying or mutating one or more genes). Cells with modified nuclease, protease or phosphatase activity (e.g., with at least one mutated phosphatase, protease or nuclease gene or combinations thereof) are especially advantageously used to increase synthesis efficiency. In an embodiment, an in vitro synthesis system has one or more polymerases modified (or polymerase activity is modulated). For one aspect, polymerase activity is reduced or substantially reduced or inhibited (for example by mutating a polymerase gene or its regulatory elements) to maintain the template. For example, RNAs bound to ribosomes are protected from RNase E. However, when the polymerase activity is too great, then the RNase E can degrade the RNA before a ribosome can bind. Thus, the template can be maintained by inhibiting RNA polymerase or by using cells with reduced polymerase activity. A cell with an RNA polymerase modified so that the polymerization speed is modulated to improve ribosome binding can be used to produce a protein synthesis system where RNA is more efficiently utilized. Also, DNA polymerases may not be needed in some systems (e.g., translation systems) and thus an embodiment relates to inhibition, reduction, substantial reduction or elimination of one or more polymerases such as a DNA polymerase, for example to prevent such DNA polymerase from utilizing reaction components (e.g., prevent unnecessary consumption of energy).

Alternatively, the cell for producing the extract can be grown under conditions such that a product, protein or activity is reduced or modulated or eliminated (e.g., to reduce expression of a product, protein or activity of interest). Such growth conditions are considered a type of modulation or inhibition. The resulting extracts will be similar to extracts obtained from cells where a gene is mutated or modified.

The cell extract can be made from any suitable cells. Suitable cells are those that have components for protein or biological macromolecules synthesis, optionally produced under selected growth conditions or with modification(s) and/or mutation(s) that inactivate or reduce or substantially reduce unwanted properties detrimental to in vitro protein or biological macromolecules synthesis. Host cells that can be used include, but are not limited to, bacterial cells, fungal and yeast cells, plant cells and animal cells. Bacterial host cells include, for example, gram positive and gram negative bacteria, and for example, Escherichia spp. cells (Escherichia coli (E. coli) cells and E. coli strains A19, D10, BL21, BL21 (DE3), Rosetta, Rosetta (DE3), Codon plus E. coli (Stratagene, La Jolla, Calif.), DH10B, Stbl2, DH5α, DB3, DB3.I DB4 and DB5 (see U.S. application Ser. No. 09/518,188, filed on Mar. 2, 2000, and U.S. Provisional Application No. 60/122,392, filed on Mar. 2, 1999, the disclosures of which are incorporated by reference herein in their entirety), HB101, INV110, INVαF′, MC1061/P3, PIR1, PIR2, TOP10, TOP10F′, TOP10/P3, Bacillus spp. cells (particularly B. subtilis and B. megaterium cells), Aspergillus spp. cells, Streptoinyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Animal host cells include insect cells (for example, Drosophila melanogaster cells and S2 cells, Spodoptera frugiperda Sf9 and Sf21 cells and Trichoplusa High-Five cells), nematode cells (particularly C. elegans cells), avian cells, amphibian cells (for example, Xenopus laevis cells), reptilian cells, and mammalian cells (NIH3T3, CHO, COS, C127, VERO, BHK, HeLa, 293, Per-C6, Bowes melanoma, and human, rabbit, mouse, rat, hamster, pig, bovine and gerbil cells generally). Yeast host cells include, for example, Saccharomyces cerevisiae cells and Pichia pastoris cells. These and other suitable host cells are available commercially, for example, from Invitrogen Corp. (Carlsbad, Calif.), Novagen (Madison, Wis.), Stratagene (La Jolla, Calif.), American Type Culture Collection (Manassas, Va.), and Agricultural Research Culture Collection (NRRL; Peoria, Ill.), the catalogues of each being incorporated in their entirety by reference. Plant cells are exemplified by protoplasts, tobacco, potato and other tuberous plants, grasses including maize, cotton and other fibrous plants, annuals and perennials, monocots and dicots, and especially plant cells that can be transformed and/or grown in culture.

The cell extract can be supplemented to provide components not present or not present in sufficient quantities after extraction. The extract can be prepared by any method used in the art that maintains the integrity of the transcription/translation system or if the process damages one or more component necessary for any stage of transcription/translation, the damaged component can be replaced or substituted for after the extract preparation.

In one embodiment extracts were prepared according to the method of, for example, Lederman and Zubay, 1967, Biochim. Biophys. Acta, 149: 253. The ordinarily skilled artisan will recognize that many modifications to the extraction process are possible within the scope of the present invention.

In a detailed embodiment of the in vitro protein and nucleic acid synthesis system, the incubation reaction contained 57 mM Hepes/KOH pH 7.6, 230 mM potassium glutamate, 80 mM ammonium acetate, 2 mM each of ATP and GTP, 0.85 mM each of UTP, CTP, 30-40 mM 3-phosphoglycerate (3-PGA), 1.7 mM DTT, 12 mM Mg (OAc)₂, 0.17 mg/ml E. coli total RNA mixture, 34 μg/ml folinic acid, 30-50 μg/ml T7 RNA polymerase, 1 mM each of amino acids, 10-20 mg/ml extract, 2% polyethylene glycol, 0.65 mM cAMP and various amount of DNA template known to one skilled in the art. The molar amount of a DNA template in the reaction mixture may vary by up to 100-fold or more depending upon whether a supercoiled plasmid DNA template is used or a linear PCR DNA template is used. For example, the molar amount of a linear PCR DNA template may be typically 10-fold, 100-fold, or more compared to the molar amount of a supercoiled plasmid DNA template. In a further detailed embodiment the incubation reaction mixture may or may not also contain 30-40 mM PEP. In all embodiments, the incubation reaction mixture excludes exogenously added enzymes or exogenously added enzymatic cofactors.

The skilled artisan will recognize that concentrations of the various components of the incubation medium can be adjusted as is known in the art while still maintaining the synthetic function. For example, the pH can range from about 5.6 to about 8.8 or more, from about 6.1 to 8.5, or from about 7.2 to 8.4 depending on the product to be synthesized. Likewise the concentration of potassium glutamate can be varied between about 80 mM and 320 mM or more, between about 120 to 280 mM or between about 180 to 250 mM; the concentration of ATP and GTP can be varied from about 0.1 to 3.0 mM or more, from about 0.3 to 2.0 mM or from about 0.8 to 1.5 mM; the concentrations of UTP and CTP can vary from about 0 or 0.1 to 2 mM or more, from about 0.4 to 1.2 mM, or from about 0.7 to 1.0 mM; 3-phosphoglycerate (3-PGA) can vary from about 10 to 80 mM or more, from about 20 to 60 mM, or from about 25 to 40 mM; phosphoenol pyruvate (PEP) can vary from about 10 to 60 mM or more, from about 20 to 50 mM, or from about 25 to 40 mM; DTT can vary from about 0.5 to 3.0 mM or more, from about 1.0 to 2.5 mM, or from about 1.5 to 2.0 mM; magnesium can vary from about 7.5 to 20 mM or more, from about 10 to 15 mM, or from about 11.5 to 14 mM; total RNA mixture can vary from about 0.1 to 0.5 mg/ml or more, from about 0.12 to 0.3 mg/ml or from about 0.15 to 0.2 mg/ml; folinic acid can vary from about 15 to 50 μg/ml or more, from about 25 to 40 μg/ml, or from about 30 to 35 μg/ml; T7 polymerase can vary from about 0 or 1.0 to 300 μg/ml or more, from about 20 to 200 μg/ml, from about 30 to 150 μg/ml, from about 40 to 120 μg/ml, from about 50 to 100 μg/ml or from about 60 to 75 μg/ml; amino acids can vary independently from about 0.4 to 5 mM or more, from about 0.5 to 2.0 mM, or from about 0.8 to 1.5 mM; extract can vary from about 2 to 40 mg protein/ml or more, from about 5 to 25 mg protein/ml or from about 10 to 20 mg protein/ml; and a synthesis marker, if desired, e.g., ³⁵S Met, or a functional protein marker, e.g., CAT or GFP) can be selected as desired and used in any amount detectable that does not unduly compromise protein or biological macromolecule synthesis. The skilled artisan will readily recognize that many of the components have known substitutes or equivalents that might be used either in the same concentration or in a concentration that produces a qualitatively and/or quantitatively similar effect.

Kits for Use in Diagnostic and/or Prognostic Applications.

For use in diagnostic, research, and therapeutic applications suggested above, kits are also provided. In the diagnostic and research applications, kits for in vitro transcription translation (IVTT) or cell free transcription translation (CFTT) systems can contain any number or combination of reagents or components. Kits for IVTT systems comprise one or more elements selected from the group consisting of one or more components (e.g., energy sources, cell extracts, inhibitors) one or more nucleotides or derivatives thereof, one or more amino acids or derivatives thereof, one or more polymerases, one or more enzymes, one or more cofactors, one or more buffers or buffer salts, one or more energy sources, one or more cell extracts (the cell extracts providing components selected from enzymes, ribosomes, transcription factors, translation factors or co-factors), one or more nucleic acid templates, molecular chaperones or foldases, lipids, cholesterol, or membranes, one or more reagents to determine the efficiency of the kit or assay for production of the products such as nucleic acid and protein products, and directions or protocols for carrying out the methods or to use of the kits and/or its components. A therapeutic product may include sterile saline or another pharmaceutically acceptable emulsion and suspension base. The kits can comprise one or more of the above components in any number of separate containers, tubes, vials and the like or such components can be combined in various combinations in such containers.

In addition, the kits for IVTT systems can include instructional materials containing directions (i.e., protocols) for the practice of the methods. While the instructional materials typically comprise written or printed materials they are not limited to such. Any medium capable of storing such instructions and communicating them to an end user is contemplated. Such media include, but are not limited to electronic storage media (e.g., magnetic discs, tapes, cartridges, chips), optical media (e.g., CD ROM), and the like. Such media may include addresses to internet sites that provide such instructional materials. A wide variety of kits and components for the IVTT system can be prepared depending upon the intended user of the kit and the particular needs of the user.

Pharmaceutical Compositions and Vaccines

Within certain aspects, polypeptides, polynucleotides and/or binding agents produced by the in vitro protein or biological macromolecule synthesis system as described herein can be incorporated into pharmaceutical compositions or immunogenic compositions (i.e., vaccines). Pharmaceutical compositions comprise one or more such compounds and a physiologically acceptable carrier. Vaccines can comprise one or more such compounds and a non-specific immune response enhancer. A non-specific immune response enhancer can be any substance that enhances an immune response to an exogenous antigen. Examples of non-specific immune response enhancers include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Vaccines can be designed to generate antibody immunity and/or cellular immunity such as that arising from CTL or CD4⁺ T cells.

Pharmaceutical compositions and vaccines can also contain other compounds, which can be biologically active or inactive. For example, one or more immunogenic portions of other tumor antigens can be present, either incorporated into a fusion polypeptide or as a separate compound, within the composition or vaccine. Polypeptides can, but need not, be conjugated to other macromolecules as described, for example, within U.S. Pat. Nos. 4,372,945 and 4,474,757. Pharmaceutical compositions and vaccines can generally be used for prophylactic and therapeutic purposes.

It will be apparent that a vaccine can contain pharmaceutically acceptable salts of the polypeptides, polynucleotides, and/or binding agents produced by the in vitro protein or biological macromolecules synthesis system as described herein. Such salts can be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art can be employed in the pharmaceutical compositions, the type of carrier will vary depending on the mode of administration. Compositions can be formulated for any appropriate manner of administration, including for example, topical, oral, nasal, intravenous, intracranial, intraperitoneal, subcutaneous or intramuscular administration. For parenteral administration, such as subcutaneous injection, the carrier comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, can be employed. Biodegradable microspheres (e.g., polylactate polyglycolate) can also be employed as carriers for the pharmaceutical compositions. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

Such compositions can also comprise buffers (e.g., neutral buffered saline or phosphate buffered saline), carbohydrates (e.g., glucose, mannose, sucrose or dextran), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions can be formulated as a lyophilizate. Compounds can also be encapsulated within liposomes using well known technology.

Any of a variety of non-specific immune response enhancers can be employed in the therapeutic compounds or vaccines. For example, an adjuvant can be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, can also be used as adjuvants.

Within the therapeutic compounds or vaccines provided herein, the adjuvant composition is designed to induce an immune response predominantly of the Th1 type. High levels of Th1-type cytokines (e.g., IFN-γ, TNF-α, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a one embodiment, in which a response is predominantly Th1-type, the level of Th1-type cytokines will increase to a greater extent than the level of Th2-type cytokines. The levels of these cytokines can be readily assessed using standard assays. For a review of the families of cytokines, see e.g., Mosmann and Coffman, 1989, Ann. Rev. Immunol. 7:145-173.

Adjuvants for use in eliciting a predominantly Th1-type response include, for example, a combination of monophosphoryl lipid A, 3-de-O-acylated monophosphoryl lipid A (3D-MPL), together with an aluminum salt. MPL adjuvants are available from Ribi ImmunoChem Research Inc. (Hamilton, Mont.; See e.g., U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555 and WP 99/33488. Immunostimulatory DNA sequences are also described, for example, by Sato et al., 1996, Science 273:352. Another adjuvant is a saponin, or QS21 (Aquila, United States), which can be used alone or in combination with other adjuvants. For example, an enhanced system involves the combination of a monophosphoryl lipid A and saponin derivative, such as the combination of QS21 and 3D-MPL as described in WO 94/00153, or a less reactive composition where the QS21 is quenched with cholesterol, as described, for example, in WO 96/33739. Other formulations comprise an oil-in-water emulsion and tocopherol. A particularly potent adjuvant formulation involving QS21, 3D-MPL and tocopherol in an oil-in-water emulsion is described, for example, in WO 95/17210.

Other adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Ribi ImmunoChem Research Inc., Hamilton, Mont.), RC-529 (Ribi ImmunoChem Research Inc., Hamilton, Mich.) and Aminoalkyl glucosaminide 4-phosphates (AGPs).

Any vaccine provided herein can be prepared using well known methods that result in a combination of antigen, immune response enhancer and a suitable carrier or excipient. The compositions described herein can be administered as part of a sustained release formulation (i.e., a formulation such as a capsule or sponge that effects a slow release of compound following administration). Such formulations can generally be prepared using well known technology (see, e.g., Coombes et al., 1996, Vaccine 14:1429-1438.) and administered by, for example, oral, rectal or subcutaneous implantation, or by implantation at the desired target site. Sustained-release formulations can contain a polypeptide, polynucleotide or antibody dispersed in a carrier matrix and/or contained within a reservoir surrounded by a rate controlling membrane.

Carriers for use within such formulations are biocompatible, and can also be biodegradable. The formulation provides a relatively constant level of active component release. Such carriers include microparticles of poly (lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles can be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that can be engineered to be efficient APCs. Such cells can, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs can generally be isolated from any of a variety of biological fluids and organs, including tumor and peritumoral tissues, and can be autologous, allogeneic, syngeneic or xenogeneic cells.

Certain embodiments use dendritic cells or progenitors thereof as antigen-presenting cells. Dendritic cells are highly potent APCs (see e.g., Banchereau and Steinman, 1998, Nature 392: 245-251) and have been shown to be effective as a physiological adjuvant for eliciting prophylactic or therapeutic antitumor immunity. See e.g., Timmerman and Levy, 1999, Ann. Rev. Med. 50: 507-529. In general, dendritic cells can be identified based on their typical shape (stellate in situ, with marked cytoplasmic processes (dendrites) visible in vitro), their ability to take up process and present antigens with high efficiency and their ability to activate naïve T cell responses. Dendritic cells can, of course, be engineered to express specific cell-surface receptors or ligands that are not commonly found on dendritic cells in vivo or ex vivo, and such modified dendritic cells are contemplated. As an alternative to dendritic cells, secreted vesicles antigen-loaded dendritic cells (called exosomes) can be used within a vaccine (See e.g., Zitvogel et al., 1998, Nature Med. 4:594-600).

Dendritic cells and progenitors can be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells can be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow can be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

Vaccines and pharmaceutical compositions can be presented in unit-dose or multi-dose containers, such as sealed ampoules or vials. Such containers are hermetically sealed to preserve sterility of the formulation until use. In general, formulations can be stored as suspensions, solutions or emulsions in oily or aqueous vehicles. Alternatively, a vaccine or pharmaceutical composition can be stored in a freeze-dried condition requiring only the addition of a sterile liquid carrier immediately prior to use.

The compositions are formulated as sterile, substantially isotonic and in full compliance with all Good Manufacturing Practice (GMP) regulations of the U.S. Food and Drug Administration.

The following examples are put forth so as to provide those of ordinary skill in the art with a complete disclosure and description of how to make and use the subject invention, and are not intended to limit the scope of what is regarded as the invention.

### EXAMPLES

### Example 1

### Escherichia coli Strains for Cell-Free Protein Synthesis

E. coli K12 (strain A19), E. coli BL21 Rosetta™, Novagen, Madison, Wis.), or E. coli D10 or derivatives thereof were used in the in vitro transcription translation (IVTT) protein synthesis system. The isogenetic strains E. coli A19 and E. coli D10 (rma-10, relA1, spoT1, metB1) have been used in the cell-free protein synthesis system (See e.g., Kudlicki et al. Analytical Biochemistry 1992, 206: 389-393; Thorson et al. 1998, Methods in Mol. Biol./Protein Synthesis: Methods and Protocols, 77: 43-73). E. coli BL21 (Rosetta™ Novagen, Madison, Wis.) strain is devoid of OmpT and lon proteases (See e.g., Studier, 1990, Methods in Enzymology, 185: 60-89, and provides extra E. coli t-RNAs cloned in pACYC184 plasmid. These extra E. coli rare t-RNAs in the extract help expressing proteins from DNA templates of non-E. coli origin containing rare codons. E. coli cells were grown in YTPG medium (See e.g., Kim and Choi, 2000, J. Biotech, 84, 27-32). E. coli S30 extract were prepared according to, e.g., Zubay, 1973, Ann Rev. Genet., 7: 267-286, except the cells were disrupted in a high-pressure (15,000-20,000 psi) C-5 high-pressure homogenizer (Avestin, Inc., Ottawa, Canada). Since the E. coli extract contains several phosphatases and proteases, in an embodiment the extract was supplemented with cocktails of both phosphatases inhibitor as well as proteases inhibitor (Roche) to stabilize the phosphatase-sensitive components and proteins. Phosphoenol pyruvate, PEP (See e.g., Kim and Swartz, 1999, Biotech. Bioeng. 66: 180-188) and ATP (See e.g., Kawaraski et al., 1994, Biosci. Biotech. Biochem, 58: 1911-1913; Kang et al. 2000, J. Microb. Meth. 43: 91-96; Kim and Choi, 2000, J. Biotech. 84: 27-32) have been reported to be depleted in the cell-free systems. Thus, in the presence of phosphatases and proteases inhibitors in the cell-free systems, the loss of ATP and also PEP (ATP regenerating substrate) can be prevented or minimized and at the same time, protein yield can be maximized.

E. coli BL21 host strains (Rosetta™, Novagen, Madison, Wis.) enhance the expression of eukaryotic proteins that contain codons rarely used in E. coli. These strains supply tRNAs for AGG, AGA, AUA, CUA, CCC, GGA codons on a compatible chloramphenicol-resistant plasmid. Thus the Rosetta strains provide for “universal” translation which is otherwise limited by the codon usage of E. coli. The tRNA genes are driven by their native promoters. In Rosetta(DE3)pLysS and Rosetta(DE3)pLacI, the rare tRNA genes are present on the same plasmids that carry the T7 lysozyme and lac repressor genes, respectively.

A mutant derivative of BL 21 (BL21 Star; Invitrogen, Carlsbad, Calif.; containing a carboxy terminal deletion of RNase E) was also used in the cell-free protein synthesis system. See, for example, PCT International Application WO 02/072890, incorporated herein by reference. The following genes were mutated: RNase I, the carboxy terminus deletion of RNase E and endonuclease I. A further derivative of BL21 Star contains a plasmid expressing rare tRNAs for codons AGG, AGA, AUA, CUA, CCC, GGA. These mutations resulted in stabilizing or maintaining the DNA template and mRNA as well as stabilizing the proteins being synthesized.

### Example 2

### Protein Synthesis Using a Glycolytic Intermediate as an Energy Source

In one aspect, an ATP regeneration system has been developed as an energy source for use during cell-free protein synthesis. In an embodiment, an in vitro protein or biological macromolecule synthesis system comprises one or more energy sources providing chemical energy for protein or biological macromolecule synthesis, wherein at least one of the energy sources is a glycolytic intermediate, and wherein the glycolytic intermediate provides chemical energy for protein or biological macromolecule synthesis without addition of an exogenous enzymatic cofactor. In a further embodiment the glycolytic intermediate is selected from 3-phosphoglycerate, 2-phosphoglycerate, 2,3-diphosphoglycerate, or 1,3-diphosphoglycerate (Sigma-Aldrich Corp., St. Louis, Mo.).

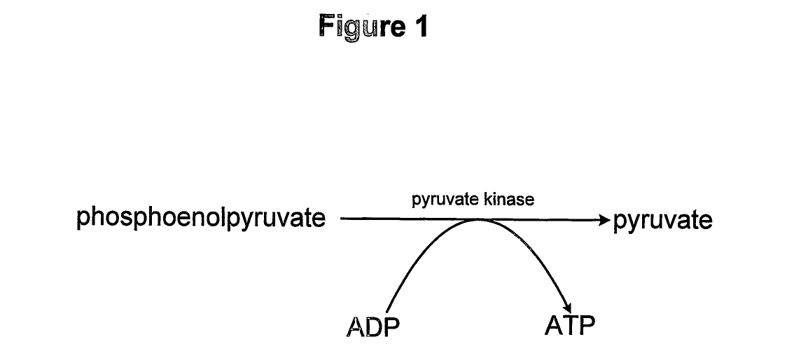

In E. coli cell-free protein synthesis systems known in the art, phosphoenol pyruvate (PEP) is used as ATP regeneration substrate. In some cases, acetyl phosphate and creatine phosphate are also used. The PEP is converted to pyruvate by pyruvate-kinase and in turn, ATP is synthesized which is then used for protein synthesis (see FIG. 1). However, the protein synthesis in the cell-free systems ceased abruptly within about 30 min. Although, the reasons for the early cessation of protein synthesis have been unclear, it has been shown that unproductive depletion of PEP can be one of the possible reasons. In fact, almost 70% depletion of PEP was shown to be dephosphorylated after 30 min by phosphatases present in the S30 extract (See e.g., Kim and Swartz, 1999, Biotech. Bioeng. 66: 180-188). The result is similar for acetyl phosphate and creatine phosphate as an energy source. Thus, this expression-independent consumption/depletion of PEP led to the production of low amount of protein synthesis due to unavailability of PEP to generate ATP in a sustained manner.

The amount of PEP added to the E. coli cell-free protein synthesis system known in the art is about 3040 mM. This high concentration of PEP can be present above the Kₘ for the phosphatases. In other words, if one can keep the concentration of PEP below the Kₘ during the course of protein synthesis, the phosphatases will not act on PEP as efficiently and thus, and PEP might be used almost exclusively by pyruvate kinase to produce ATP. There are different ways to stabilize PEP or control the action of the phosphatases. First, one can grow the culture in a high phosphate medium (See e.g., Kim and Choi, 2000, J. Biotech. 84: 27-32). It has been reported that growth of E. coli cells in high phosphate containing media represses the synthesis of alkaline phosphatases (See e.g., Malamy and Horecker, 1964, Biochemistry 3: 1893-1897). Second, it is possible to inject/infuse PEP, as well as other components, slowly by dialysis (See e.g., Spirin et al. 1988, Science 242: 1162-1164) or other means. Third, pyruvate and NAD/Acetyl CoA mixture can be used instead of PEP as ATP regenerating system (See e.g., Kim and Swartz, 1999, Biotech and Bioeng. 66: 180-188 and Kim and Swartz, 2001, Biotech and Bioeng. 74: 309-316). Fourth, use an E. coli completely devoid (mutants) of all phosphatases, however, some phosphatases activities are required for cell maintenance.

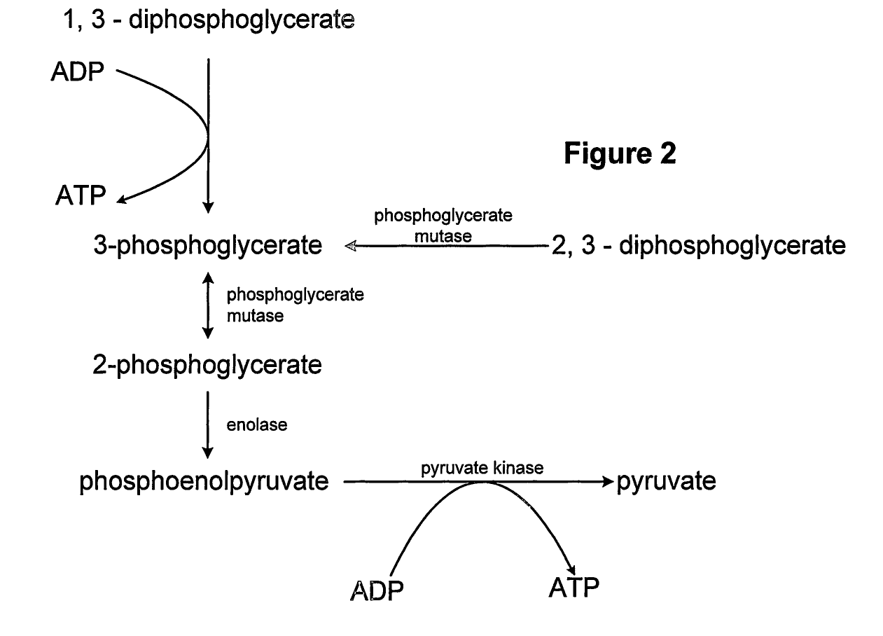

The in vitro protein synthesis system utilized a glycolytic intermediate without the addition of an exogenous enzyme, for example, NAD⁺, for energy regeneration in the form of ATP synthesis. The glycolytic intermediate, 3-phosphoglycerate (3-PGA), was used to generate PEP in situ using two endogenous enzymes, phosphoglycerate mutase and enolase. Since PEP was not present in the extract at the beginning, PEP will be produced in situ by two enzymatic reactions sequentially and continually from 3-PGA (FIG. 2). The conversion of PEP to pyruvate and ATP is coupled to PEP synthesis. In addition, the concentration of PEP at any given time will be low enough (below Kₘ) so that the phosphatases will not target PEP as substrate. The fact that the conversion of PEP to pyruvate and ATP is coupled to PEP synthesis will result in sustained production of ATP during protein synthesis for an extended period of time.

The glycolytic intermediate, 3-PGA, as an energy regenerating system, is added to the modified reaction mixture for the cell-free protein synthesis system. The standard reaction mixture has been described, e.g., in Kim and Choi, 1996, Biotechnol. Prog. 12: 645-649. Nucleotides, phosphoenolpyruvate (PEP), and E. coli total tRNA mixture were purchased from Boehringer-Mannheim. 3-phosphoglycerate, 2-phosphoglycerate, and 2,3-diphosphoglycerate were purchased from Sigma-Aldrich Corp., St. Louis, Mo. Dialysis membrane (MW cutoff=8,000-10,000) was purchased from Pierce Inc (Illinois). T7 RNA polymerase is available from several commercial sources (US Biochemical, Inc., Invitrogen, Inc.). As a template for the cell-free coupled transcription/translation reaction, a chloramphenicol acetyl transferase (CAT) gene sequence under the control of T7 promoter can be used. Alternatively, a green fluorescent protein (GFP) sequence under the control of T7 promoter can be used.

E. coli strains A19, Rosetta (DE3) and BL21 Star containing the rare t-RNA genes were used for S30 extract preparation. Cells were grown at 37° C. in 15 L of 2×TYPG (Kim and Choi, 2000, J. Biotech. 84: 27-32) medium with vigorous agitation and aeration. Cells were harvested in mid-log phase and used for subsequent S3o preparation with minor modifications. See for example, Zubay (Ann Rev. Genet. 1973, 7: 267-286), and Pratt, 1984, Coupled Transcription-Translation In Prokaryotic Cell-Free Systems, 179-209. Unless otherwise specified, the reaction mixture contains 57 mM HEPES-KOH (pH 7.6), 2 mM each of GTP and ATP, 0.85 mM each of UTP, and CTP, 1.7 mM DTT, 0.65 mM cAMP, 208 mM potassium glutamate, 80 mM NH₄ (OAc), 12 mM Mg (OAc)₂, 0.17 mg/m E. coli total tRNA mixture, 34 mg/mL folinic acid, various amount of DNA template (DNA expression vector for a gene of interest, either supercoiled plasmid DNA or linear PCR DNA; See detailed description above.), 30-50 μg/mL T7 RNA polymerase, 1 mM each of 20 unlabeled amino acids (Cysteine was present at 2 mM), 2% PEG, 30-40 mM 3-PGA, and 15-20 ul of S30 extract (20-35 mg/ml) in a total volume of 50 μl. Reactions were carried out between about 30° C. and about 37° C. See e.g., Kim and Choi, 1996, Biotechnol. Prog. 12: 645-649.

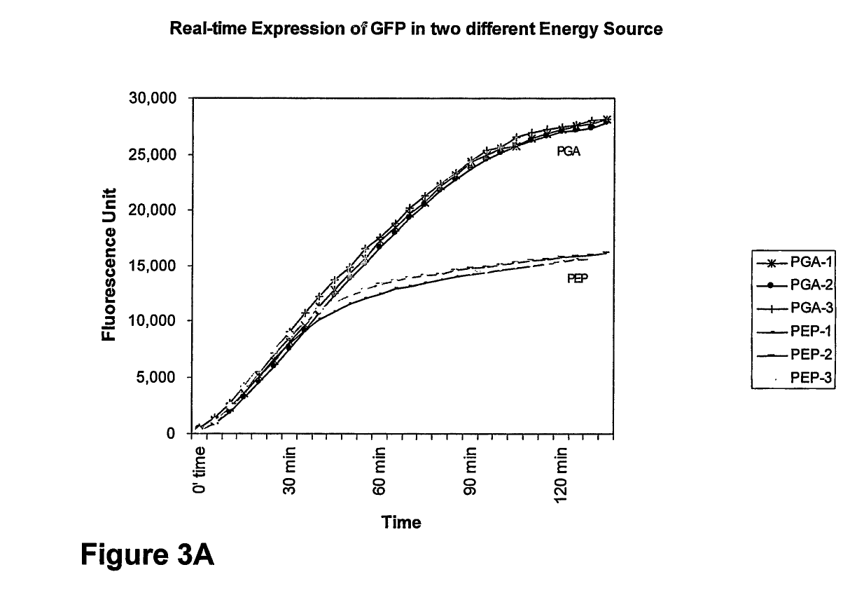

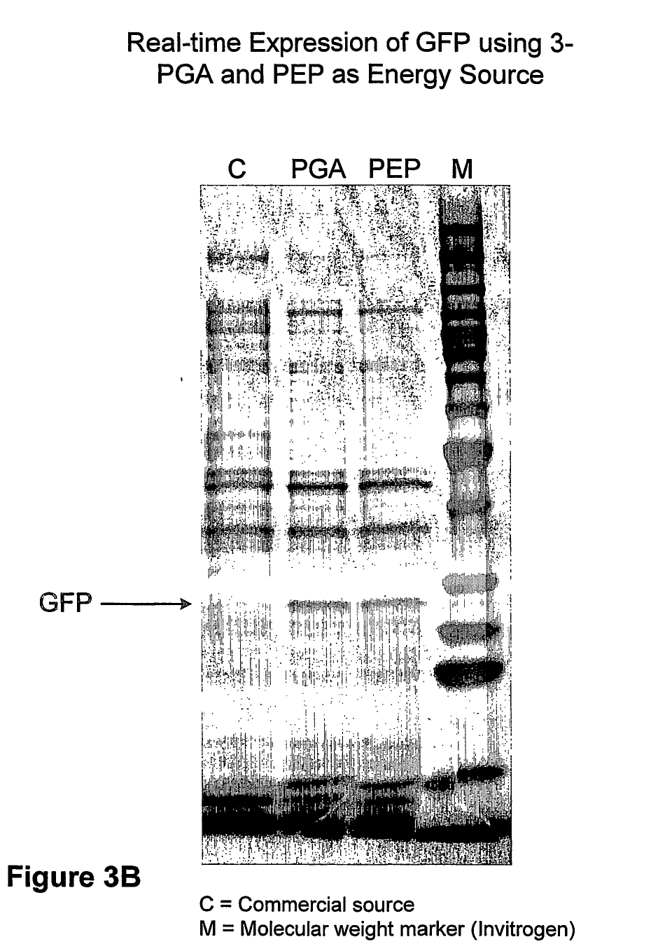

The composition of standard reaction mixture for the cell-free protein synthesis system is similar as described above. See e.g., Kim and Choi, 1996, Biotechnol. Prog. 12: 645-649. The following changes were made for the exemplary embodiment: ATP and GTP were present at 2 mM, 3-PGA was present at 40 mM, amino acids were present at 1.0 mM. See, e.g., Kigawa et al. FEBS lett. 1999, 442: 15-19. Cysteine was present at 2.0 mM. No exogenous enzymatic cofactor was added to the IVTT system of the exemplary embodiment. No exogenous pyruvate kinase or other exogenous enzyme was added to the to the IVTT system of the exemplary embodiment. A typical reaction was carried out in 50 μl total at 30° C. for 1-3 hours unless otherwise stated. At the end of the reaction, 0.5-1 μl of the reaction was loaded on to a discontinuous SDS-polyacrylamide gel electrophoresis (Invitrogen Corp., Carlsbad, Calif.; or BioRad Corp., Hercules, Calif.) according to the method, e.g., of Laemmli, 1970, Nature 227: 680-685. Real time expression of green fluorescent protein (GFP) utilizing 3-PGA compared to PEP as an energy source is shown in FIGS. 3A and 3B.

Using 3-PGA as an energy regenerating system in an exemplary embodiment, in vitro protein synthesis as measured by synthesis of green fluorescent protein (GFP) continued beyond 2 hours. When PEP was used as an energy source in a control reaction, GFP synthesis halted within 45 minutes (FIG. 3a). FIG. 3a follows the synthesis of GFP in the IVTT system in real-time. In addition, the yield of GFP protein was 2-fold greater when 3-PGA was used as an energy regenerating system compared to the yield of GFP protein generated from an equivalent molar concentration of PEP (FIG. 3b).

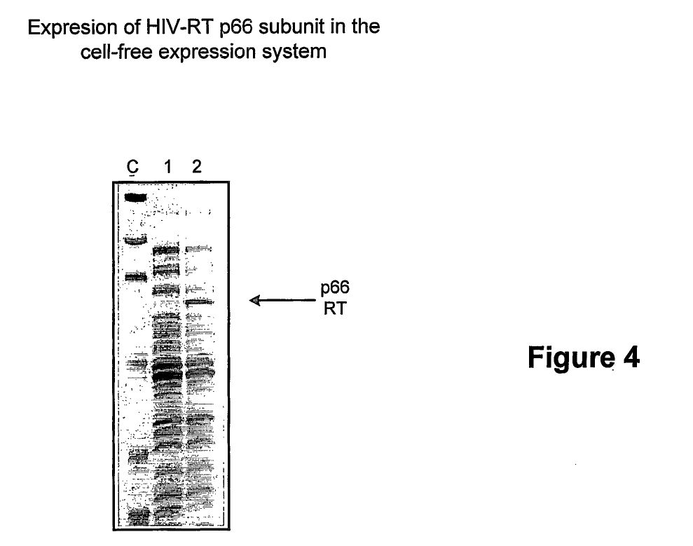

To further examine the potential of this exemplary energy regenerating system, the expression of HIV-reverse transcriptase (HIV-RT) in an exemplary IVTT system utilizing 3-PGA as an energy regenerating system was compared to a commercially available system utilizing phosphoenolpyruvate (PEP). As can be seen from the FIG. 4, the expression of HIV-RT was almost 5-6 fold better in the IVTT system utilizing 3-PGA as an energy regenerating system in an exemplary embodiment compared to the IVTT system utilizing PEP. The expression of HIV-RT in the IVTT system utilizing 3-PGA as an energy regenerating system in an exemplary embodiment was equivalent to an in vivo E. coli expression system induced to express p66 RT with IPTG.

### Example 3

### Enhanced Expression of Proteins in the Presence of Lipids

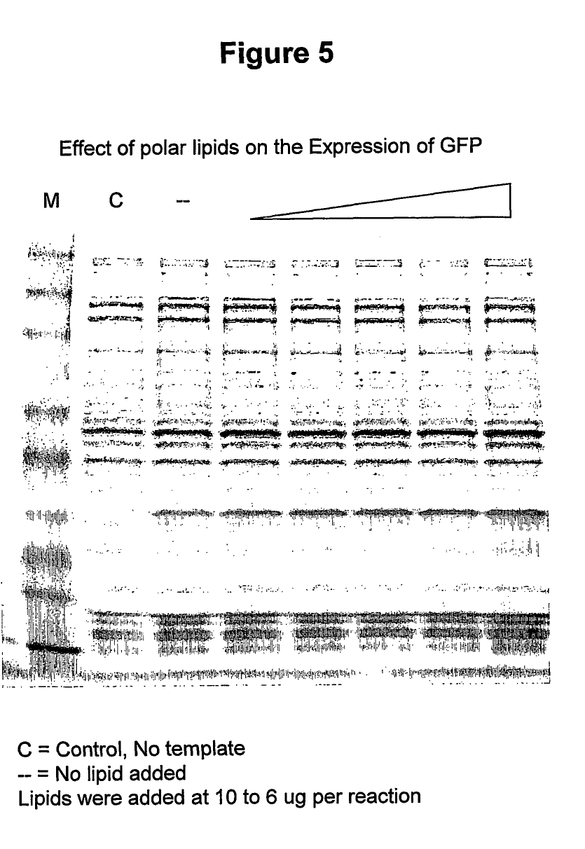

During preparation of E. coli extract for cell-free protein synthesis, the membranes are generally removed during centrifugation. In some cases, particularly during synthesis of membrane-associated proteins in the cell-free system, it can be advantageous to add membranes, cholesterol, or lipids as anchors. To test production of membrane proteins, e.g., G-protein coupled receptors (GPCR), E. coli polar lipids (Avanti® Polar Lipids, Inc., Alabaster, Ala.) were tested to determine an effect on the IVTT system. The results show that E. coli polar lipids have a beneficial and dose-dependent positive effect on the production of GFP (FIG. 5) in an IVTT system of the exemplary embodiment, e.g., a reaction mixture comprising 3-PGA+cysteine.

### Example 4

### Effect of Chaperones in Soluble Expression of Otherwise Insoluble Proteins

Expression of recombinant proteins in either prokaryotic or eukaryotic IVTT systems often encounters solubility problems. The expressed protein can be insoluble or in aggregated form. It is extremely difficult to refold the proteins, particularly if the protein is high molecular weight. It is advantageous to make the protein(s) in vitro in a native conformation during its synthesis. The mechanism by which newly synthesized proteins acquires the native conformation is unclear. However, folding of proteins in the cell typically requires the assistance of other proteins, called molecular chaperones and foldases. In E. coli different classes of chaperones exist. See, for example, Schlieker et al., 2002, J. Biotech. 96: 13-21; Hartl and Hayer-Hartl, 2002, Science 295: 1852-1858. Ribosome-associated trigger factor (TF) helps folding of newly synthesized proteins. Hsp 70 (DnaK) act with co-factors (Dna J and GrpE) to recognize proteins in an extended conformation. GroEL/ES (Hsp 60) assists in the folding of compact folding intermediates. Clp ATPases (Hsp 100) can unfold and disaggregate aggregated proteins. The foldases, which accelerate rate-limiting steps along the folding pathway, include: peptidyl prolyl cis/trans isomerases (PPI's) (see e.g., Dartigalongue and Raina, 1998, EMBO J. 17: 3968-3980); Dsb (disulfide bond formation) proteins (see e.g., Wulfing and Pluckthun, 1994, J. Mol. Biol. 242: 655-669; Bardwell J. C., 1994, Mol. Micro. 14: 199-205; Georgiou and Valex, 1996, Curr. Opin. Biotechnol. 7: 190-197); FkpA which catalyze the cis/trans isomerization of prolyl peptide bonds (see e.g., Arie et al. 2001, Mol. Microbiol. 39: 199-210) and Skp (see e.g., Holck et al., 1997, Biochim. Biophys. Acta. 908: 188-199; Hayhurst and Harris, 1999, Prot. Exp. Purif. 15: 336-343). In addition, there are uncharacterized and unknown chaperones and foldases.

A cell-free transcription translation system can utilize chaperones. In vitro transcription translation synthesis of active rhodanase occurs in the presence of a combination of chaperones. See e.g., Kudlicki et. al, 1994, J. Biol. Chem. 269: 16549-16553. A cell-free protein synthesis system can be made devoid of endogenous chaperones. A chaperone-deficient system was used to study synthesis and folding of bacterial dihydrofolate reductase and of rhodanase, a eukaryotic mitochondrial enzyme. Both enzymes were synthesized in active form and with high specific activity in the chaperone-deficient system as well as in a chaperone proficient host. However, in the chaperone-deficient host, a portion of these two enzymes stayed bound (inactive) to the ribosome. Addition of GroEL and DnaK released the enzymes in an active form. See e.g., Kudlicki et al. 1994, Anal Biochem. 217:12-19.

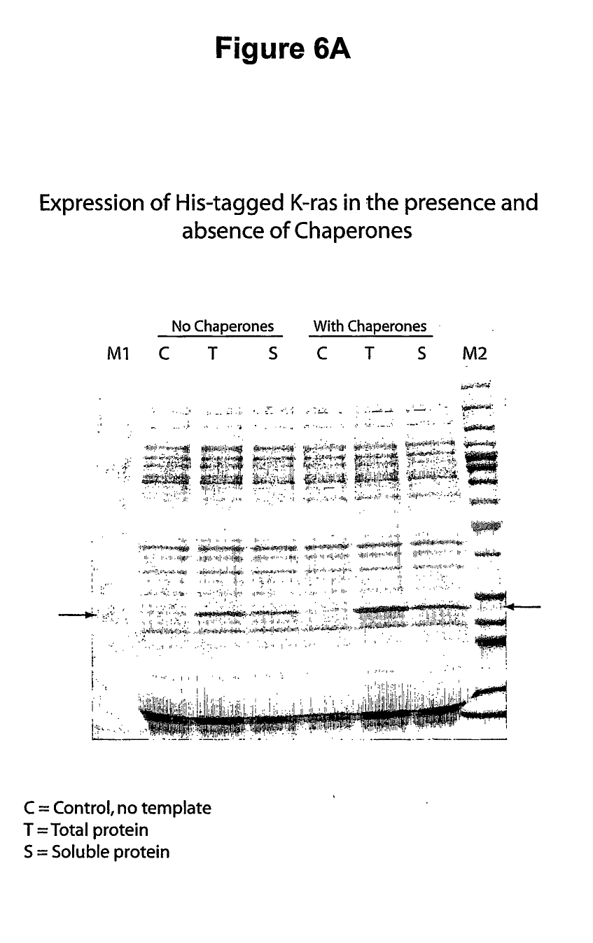

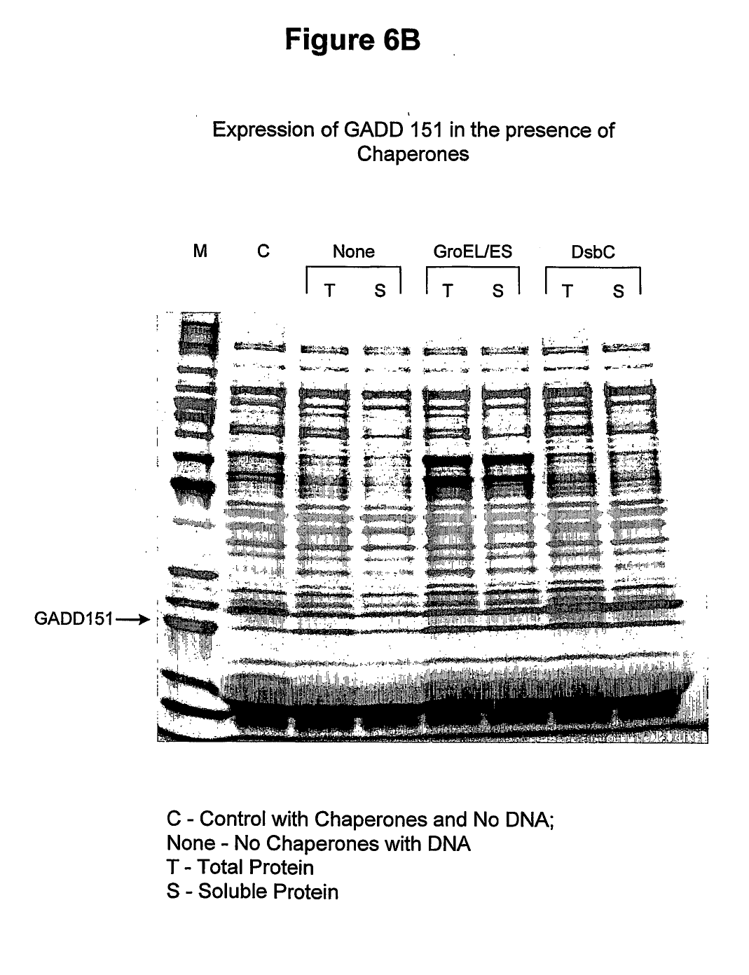

Different chaperones can work better in vitro compared to in vivo for some proteins compared to other proteins. For example, the GroEL/ES system can account for folding of no more than 5% of the proteins produced in rapidly growing E. coli. See e.g., Lorimer, 1996, FASEB 10: 5-9. In order to avoid problems of choosing a particular chaperone or a few chaperones, certain embodiments use a cocktail of chaperones and foldases in the cell free transcription translation system. In the cell free transcription translation system, a cocktail of GroEL/ES, TF, DnaK, DnaJ, GrpE, ClpB, FkpA, Skp and DsbC, improved the expression of His-tagged K-Ras. See FIG. 6A. The His-tagged K-Ras protein was produced as insoluble proteins when expressed in a cell culture in vivo or without the presence of chaperones in vitro. The presence of chaperones in the IVTT system (e.g., a reaction mixture comprising chaperones+3-PGA or chaperones+3-PGA+cysteine) improved the overall production of K-Ras. Since a IVTT system is an open system, it is possible to incorporate any number chaperones, co-factors, foldases, protease inhibitors, or other components in the system.

Molecular chaperones were added as an E. coli extract rather than purified protein. 1-2 μl of E. coli extract containing the chaperone was added. Extracts were made from chaperone over producing strains. In 1-2 μl extract, each chaperone is approximately 2 to 10 μg depending upon the type of molecular chaperone.

In a further example, chaperones added to a IVTT system are effective for improving total and soluble protein synthesis, for example, for in vitro synthesis of the mammalian protein, GADD151. GADD151 is a polypeptide subfragment of GADD34 protein a mammalian apoptosis and DNA damage-inducible gene. See e.g., Hollander et al., 1997, J. Biol. Chem. 272: 13731-13737. In an exemplary embodiment, the presence of chaperones GroEL/ES or DsbC improved expression of GADD151 in the IVTT system (+3-PGA, +chaperones) compared to a IVTT system (+3-PGA, +cysteine) in the absence of chaperones. See FIG. 6B.

### Example 5

### Expression of CAT and GFP Using Plasmid and PCR Products

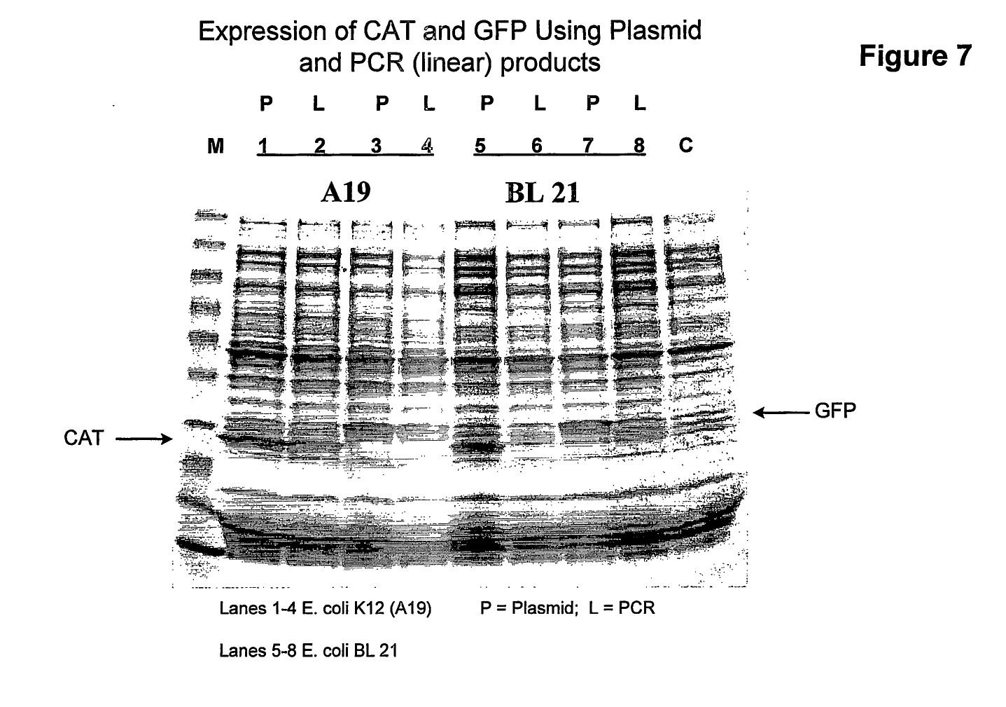

Expression of CAT and GFP proteins was demonstrated from a DNA template derived from supercoiled plasmid DNA or from a linear DNA PCR product. FIG. 7 shows synthesis of both CAT and GFP proteins in the IVTT system (+3-PGA, +cysteine) from both supercoiled plasmid DNA template and from a linear PCR DNA template utilizing E. coli cell extracts from an A19 strain or from a 13L21 strain. Synthesis of CAT and GFP proteins was improved from the supercoiled plasmid DNA template (lanes 1, 3, 5, 7) when compared to the linear DNA PCR template (lanes 2, 4, 6, 8).

### Example 6

### Expression of CAT as Linear Template in BL21 in the Presence of Gam Activity from E. coli K12

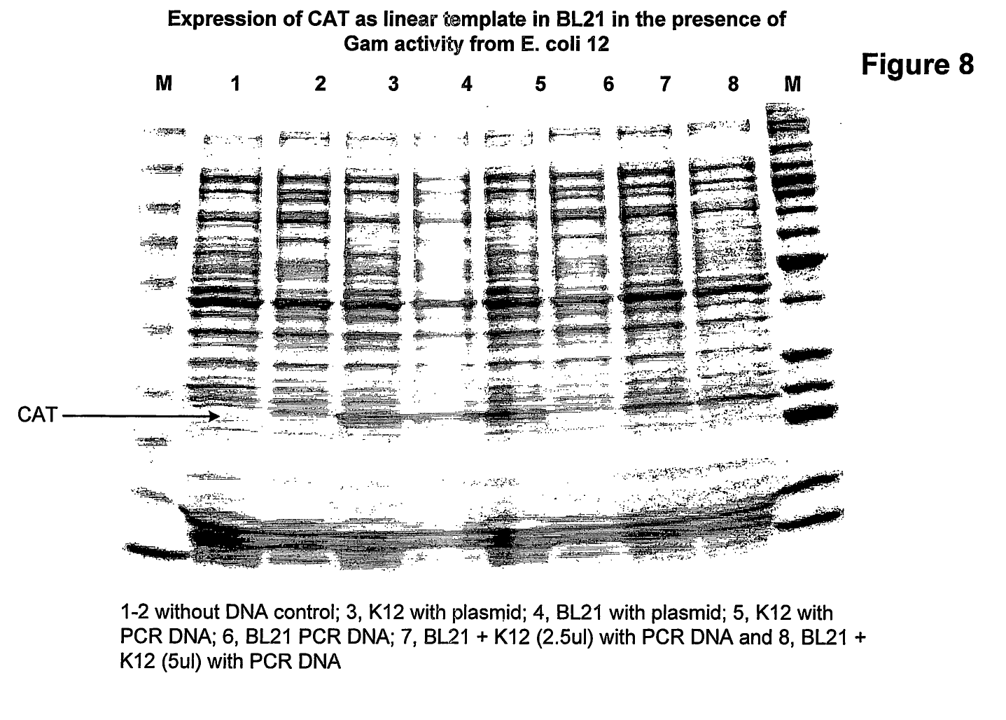

Expression of CAT as linear template in BL21 in the presence of Gam activity from E. coli K12 (A19) is shown. E. coli K12 (A19) is a lambda lysogen that releases lambda phage during growth and produces lambda Gam protein which is active to inhibit RecBCD nuclease. RecBCD nuclease is known to degrade linear DNA. FIG. 8 shows that a DNA template derived from supercoiled plasmid DNA or a linear PCR DNA showed improved synthesis of CAT protein in the presence of an E. coli K12 cell extract which provides Gam activity to inhibit the RecBCD nuclease compared to an E. coli BL21 cell extract which does not provide Gam activity.

### Example 7

### Incorporation of Fluoro-Tyrosine into GFP and CAT

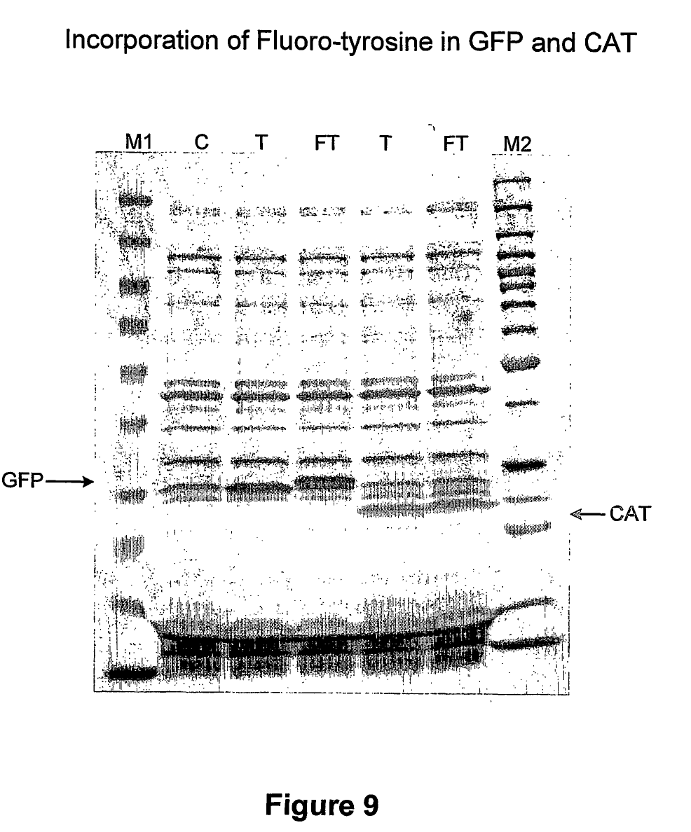

Incorporation of fluoro-tyrosine into GFP and CAT utilizes an exemplary embodiment of the IVTT system (+3-PGA, +cysteine). Fluoro-tyrosine incorporation into GFP or CAT protein is comparable to tyrosine incorporation into GFP or CAT protein. A comparison of a 19 amino acid control (C; excluding tyrosine) with 20 amino acids (T; including tyrosine) or 20 amino acids (FT; including fluoro-tyrosine) is shown in FIG. 9. A IVTT system incorporating fluoro-tyrosine into a protein of interest is useful for NMR spectrometric analysis of the protein of interest.

The IVTT system enables the incorporation of a wide variety of modified amino acids either at a predetermined site (site-directed) or at random sites within the polypeptide. For structural investigations, for example, utilizing nuclear magnetic resonance (NMR) imaging, modification of proteins with fluorine and/or selenium is extremely valuable. Modification of the protein with fluorine atom has the advantages of small size, natural abundance, sensitivity to NMR detection, and replacement of hydrogen atom in proteins with minimal structural change.

Incorporation of modified amino acids into protein utilizing an exemplary IVTT system provides tools for (1) functional analysis of proteins, and (2) analysis of protein structures by crystallization and X-ray crystallography or by NMR spectroscopy.

### Example 8

### Protein Synthesis Using a Glycolytic Intermediate and a Tricarboxylic Acid (TCA) Cycle Intermediate as Energy Sources

In another aspect, an ATP regeneration system has been developed as an energy source for use during cell-free protein synthesis. In an embodiment, an in vitro protein or biological macromolecule synthesis system comprises one or more energy sources providing chemical energy for protein or biological macromolecule synthesis, wherein at least one of the energy sources is a glycolytic intermediate and wherein at least one of the energy sources is a tricarboxylic acid (TCA) cycle intermediate, and wherein the glycolytic intermediate and the TCA cycle intermediate further in combination with an exogenous enzymatic cofactor provide chemical energy for protein or biological macromolecule synthesis. In a further embodiment the glycolytic intermediate is selected from 3-phosphoglycerate, 2-phosphoglycerate, 2,3-diphosphoglycerate, or 1,3-diphosphoglycerate (Sigma-Aldrich Corp., St. Louis, Mo.). In a further embodiment the TCA cycle intermediate is selected from citrate, isocitrate, malate, oxaloacetate, α-ketoglutarate, succinate or fumarate. Sigma Aldrich Corp. St. Louis, Mo.

The TCA cycle intermediate, citrate, and the exogenous enzymatic cofactor, NAD⁺, as components of an energy regenerating system, were added to the modified reaction mixture for the cell-free protein synthesis system. The glycolytic intermediate, 3-PGA, as a component of an energy regenerating system, was added to the modified reaction mixture for the cell-free protein synthesis system, as described above in Example 2. The standard reaction mixture has been described, e.g., in Kim and Choi, 1996, Biotechnol. Prog. 12: 645-649. Nucleotides, phosphoenolpyruvate (PEP), and E. coli total tRNA mixture were purchased from Boehringer-Mannheim. 3-phosphoglycerate, 2-phosphoglycerate, and 2,3-diphosphoglycerate were purchased from Sigma-Aldrich Corp., St. Louis, Mo. Dialysis membrane (MW cutoff=8,000-10,000) was purchased from Pierce Inc (Illinois). T7 RNA polymerase is available from several commercial sources (US Biochemical, Inc., Invitrogen, Inc.). As a template for the cell-free coupled transcription/translation reaction, a chloramphenicol acetyl transferase (CAT) gene sequence under the control of T7 promoter can be used. Alternatively, a green fluorescent protein (GFP) sequence under the control of T7 promoter can be used.

E. coli strains A19, Rosetta (DE3) and BL21 Star containing rare t-RNA genes were used for S30 extract preparation. Cells were grown at 37° C. in 15 L of 2×TYPG (Kim and Choi, 2000, J. Biotech. 84: 27-32) medium with vigorous agitation and aeration. Cells were harvested in mid-log phase and used for subsequent S30 preparation with minor modifications. See for example, Zubay (Ann Rev. Genet. 1973, 7: 267-286), and Pratt, 1984, Coupled Transcription-Translation In Prokaryotic Cell-Free Systems, 179-209. Unless otherwise specified, the reaction mixture contains 57 mM HEPES-KOH (pH 7.6), 2 mM each of GTP and ATP, 0.85 mM each of UTP, and CTP, 1.7 mM DTT, 0.65 mM cAMP, 208 mM potassium glutamate, 80 mM NH₄ (OAc), 12 mM Mg (OAc)₂, 0.17 mg/mL E. coli total tRNA mixture, 34 mg/mL folinic acid, 0.4 mM NAD, 1.25 mM Coenzyme A, various amount of DNA template (DNA expression vector for a gene of interest, either supercoiled plasmid DNA or linear PCR DNA; See detailed description above.), 30-50/g/mL T7 RNA polymerase, 1 mM each of 20 unlabeled amino acids (Cysteine was present at 2 mM), 1.5 mM spermidine, 250 mM Betaine, and 2% Trehelose, 30-40 mM 3-PGA, 40 mM citrate, and 15-20 ul of S30 extract (20-35 mg/ml) in a total volume of 50 μl. Reactions were carried out between about 30° C. and about 37° C. See e.g., Kim and Choi, 1996, Biotechnol. Prog. 12: 645-649.

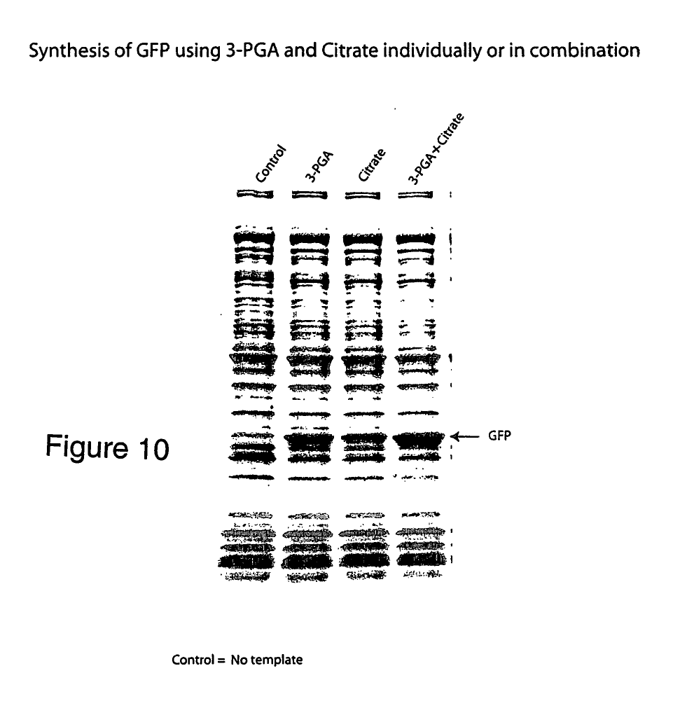

The composition of standard reaction mixture for the cell-free protein synthesis system is similar as described above. See e.g., Kim and Choi, 1996, Biotechnol. Prog. 12: 645-649. The following changes were made for this exemplary embodiment: ATP and GTP were present at 2 mM, 3-PGA was present at 40 mM, citrate was present at 40 mM, amino acids were present at 1.0 mM. Trehelose replaced polyethylene glycol (PEG) in the reaction mixture of Example 2. See, e.g., Kigawa et al. FEBS lett. 1999, 442: 15-19. Cysteine was present at 2.0 mM. An exogenous enzymatic cofactor was added to the IVTT system of the exemplary embodiment when a TCA cycle intermediate was used as a component of the energy generating or regenerating system. No exogenous pyruvate kinase or other exogenous enzyme was added to the to the IVTT system of the exemplary embodiment. A typical reaction was carried out in 50 μl total at 30° C. for 1-3 hours unless otherwise stated. At the end of the reaction, 0.5-1 μl of the reaction was loaded on to a discontinuous SDS-polyacrylamide gel electrophoresis (Invitrogen Corp., Carlsbad, Calif.; or BioRad Corp., Hercules, Calif.) according to the method, e.g., of Laemmli, 1970, Nature 227: 680-685. Real time expression of green fluorescent protein (GFP) utilizing 3-PGA compared to PEP as an energy source is shown in FIG. 10.

The presence of spermidine (1.5 mM), Betaine (250 mM), and Trehelose (2%) in place of PEG in the reaction mixture provided a quantity of protein synthesis, comparable to protein synthesis from the reaction mixture of Example 2. The addition of these components did improve the separation and resolution of proteins on the SDS polyacrylamide gel analysis, particularly below the 20 Kd molecular weight range.

Using the glycolytic intermediate, 3-PGA, the TCA cycle intermediate, citrate, and the exogenous enzymatic cofactor, NAD⁺, as an energy regenerating system in an exemplary embodiment, in vitro protein synthesis was measured by synthesis of green fluorescent protein (GFP), or synthesis of chloramphenicol acetyl transferase protein (CAT) for at least 2 hours. When 3-PGA and/or citrate was used as an energy source in an IVTT reaction, GFP synthesis was measured after 2 hour incubation. When glycolytic intermediates alone or TCA cycle intermediates alone, e.g., citrate, isocitrate, malate, oxaloacetate, α-ketoglutarate, succinate or fumarate, were used as an energy source in an IVTT reaction, comparable levels of GFP protein was synthesized. The yield of GFP protein increased 50% when the IVTT system utilized 3-PGA in combination with the TCA intermediate, citrate, as an energy regenerating system was compared to the yield of GFP protein generated from an equivalent molar concentration of 3-PGA alone (FIG. 10). Similar increased yields were observed when CAT protein was the product of the IVTT reaction.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. An in vitro protein or biological macromolecule synthesis system comprising:
one or more energy sources providing chemical energy for protein or biological macromolecule synthesis wherein at least one of said energy sources is a glycolytic intermediate.

2. The in vitro protein or biological macromolecule synthesis system according to claim 1, wherein said one or more energy sources generate or regenerate high-energy triphosphate compounds.

3. The in vitro protein or biological macromolecule synthesis system according to claim 2, wherein said high-energy triphosphate compound is adenosine triphosphate, guanosine triphosphate or a combination of adenosine triphosphate and guanosine triphosphate.

4. The in vitro protein or biological macromolecule synthesis system according to claim 1, wherein said glycolytic intermediate is selected from the group consisting of 3-phosphoglycerate, 2-phosphoglycerate, 2,3-diphosphoglycerate and 1,3-diphosphoglycerate.

5. The in vitro protein or biological macromolecule synthesis system of claim 1, wherein said glycolytic intermediate is 3-phosphoglycerate.

6. The in vitro protein or biological macromolecule synthesis system of claim 1, wherein said glycolytic intermediate provides chemical energy for biological macromolecule synthesis without addition of an exogenous enzymatic cofactor.

7. The in vitro protein or biological macromolecule synthesis system of claim 6, wherein said exogenous enzymatic cofactor is selected from the group consisting of thiamine pyrophosphate, FAD⁺, FADH, NAD⁺, NADH, NADP⁺ and NADPH.

8. The in vitro protein or biological macromolecule synthesis system of claim 6, wherein said exogenous enzymatic cofactor is selected from the group consisting of NAD⁺ and NADH.

9. The in vitro protein or biological macromolecule synthesis system of claim 1, wherein said biological macromolecule is selected from the group consisting of protein, polypeptide and ribonucleic acid.

10. The in vitro protein or biological macromolecule synthesis system of claim 1, further comprising a cellular extract to provide components selected from the group consisting of enzymes, ribosomes, transcription factors, translation factors and co-factors.

11. The in vitro protein or biological macromolecule synthesis system of claim 10, further comprising a cellular extract to provide components for E. coli rare tRNAs selected from tRNAs for amino acids selected from the group consisting of arginine, proline, glycine, leucine and isoleucine.

12. The in vitro protein or biological macromolecule synthesis system of claim 10, further comprising a cellular extract to provide components selected from the group consisting of lipids, cholesterol, and membranes.

13. The in vitro protein or biological macromolecule synthesis system according to claim 1, wherein said one or more energy sources further comprise a tricarboxylic acid (TCA) cycle intermediate.

14. The in vitro protein or biological macromolecule synthesis system according to claim 13, wherein said one or more energy sources generate or regenerate high-energy triphosphate compounds.

15. The in vitro protein or biological macromolecule synthesis system according to claim 14, wherein said high-energy triphosphate compound is adenosine triphosphate, guanosine triphosphate or a combination of adenosine triphosphate and guanosine triphsphate.

16. The in vitro protein or biological macromolecule synthesis system according to claim 13, wherein said TCA cycle intermediate is citrate, isocitrate, malate, oxaloacetate, α-ketoglutarate succinate or fumarate.

17. The in vitro protein or biological macromolecule synthesis system according to claim 16, wherein said TCA cycle intermediate is citrate.

18. The in vitro protein or biological macromolecule synthesis system according to claim 13, wherein said glycolytic intermediate is 3-phosphoglycerate, 2-phosphoglycerate, 2,3-diphosphoglycerate or 1,3-diphosphoglycerate.

19. The in vitro protein or biological macromolecule synthesis system according to claim 18, wherein said glycolytic intermediate is 3-phosphoglycerate.

20. The in vitro protein or biological macromolecule synthesis system of claim 13, wherein said TCA cycle intermediate and said glycolytic intermediate further comprise an exogenous enzymatic cofactor to generate or regenerate high-energy triphosphate compounds for biological macromolecule synthesis.

21. The in vitro protein or biological macromolecule synthesis system of claim 20, wherein said exogenous enzymatic cofactor is thiamine pyrophosphate, FAD⁺, FADH, NAD⁺, NADH, NADP⁺ or NADPH.

22. The in vitro protein or biological macromolecule synthesis system of claim 21, wherein said exogenous enzymatic cofactor is NAD⁺ or NADH.

23. The in vitro protein or biological macromolecule synthesis system of claim 13, wherein said biological macromolecule is protein, polypeptide or ribonucleic acid.

24. The in vitro protein or biological macromolecule synthesis system of claim 13, further comprising a cellular extract to provide components selected from enzymes, ribosomes, transcription factors, translation factors or co-factors.

25. The in vitro protein or biological macromolecule synthesis system of claim 24, further comprising a cellular extract to provide components for E. coli rare tRNAs selected from tRNAs for amino acids selected from arginine, proline, glycine, leucine or isoleucine.

26. The in vitro protein or biological macromolecule synthesis system of claim 24, further comprising a cellular extract to provide components selected from the group consisting of lipids, cholesterol, and membranes.

27. The in vitro protein or biological macromolecule synthesis system of claim 13, wherein said one or more energy sources is pyruvate, phosphoenolpyruvate (PEP), carbamoyl phosphate, acetyl phosphate, creatine phosphate, phosphopyruvate, 3-phosphoglycerate, 2-phosphoglycerate, 2,3-diphosphoglycerate, 1,3-diphosphoglycerate, triose phosphate, glyceraldehyde-3-phosphate, fructose-1,6-diphosphate, fructose-6-phosphate, glucose-1-phosphate, glucose-6-phosphate, or glucose, and wherein said one or more energy sources is citrate, isocitrate, malate, oxaloacetate, α-ketoglutarate, succinate or fumarate.

28. An in vitro protein or biological macromolecule synthesis system comprising:
one or more energy sources providing chemical energy for protein or biological macromolecule synthesis wherein at least one of said energy sources is a tricarboxylic acid (TCA) cycle intermediate.

29. The in vitro protein or biological macromolecule synthesis system according to claim 28, wherein said one or more energy sources generate or regenerate high-energy triphosphate compounds.

30. The in vitro protein or biological macromolecule synthesis system according to claim 29, wherein said high-energy triphosphate compound is adenosine triphosphate, guanosine triphosphate or a combination of adenosine triphosphate and guanosine triphosphate.

31. The in vitro protein or biological macromolecule synthesis system of claim 28, wherein said TCA cycle intermediate further comprises an exogenous enzymatic cofactor to generate or regenerate high-energy triphosphate compounds for biological macromolecule synthesis.

32. The in vitro protein or biological macromolecule synthesis system of claim 31, wherein said exogenous enzymatic cofactor is thiamine pyrophosphate, FAD⁺, FADH, NAD⁺, NADH, NADP⁺ or NADPH.

33. The in vitro protein or biological macromolecule synthesis system of claim 32, wherein said exogenous enzymatic cofactor is NAD⁺ or NADH.

34. The in vitro protein or biological macromolecule synthesis system of claim 1, wherein at least two energy sources are selected from the group consisting of pyruvate, phosphoenolpyruvate (PEP), carbamoyl phosphate, acetyl phosphate, creatine phosphate, phosphopyruvate, 3-phosphoglycerate, 2-phosphoglycerate, 2,3-diphosphoglycerate, 1,3-diphosphoglycerate, triose phosphate, glyceraldehyde-3-phosphate, fructose-1,6-diphosphate, fructose-6-phosphate, glucose-1-phosphate, glucose-6-phosphate and glucose.

35. The in vitro protein or biological macromolecule synthesis system of claim 1, wherein said glycolytic intermediate provides chemical energy for biological macromolecule synthesis without addition of an exogenous enzyme.

36. The in vitro protein or biological macromolecule synthesis system of claim 35, wherein said exogenous enzyme is selected from the group consisting of pyruvate kinase, acetate kinase, pyruvate oxidase, creatine kinase and creatine phosphokinase.

37. The in vitro protein or biological macromolecule synthesis system according to claim 1, further comprising adenosine triphosphate and cysteine.

38. The in vitro protein or biological macromolecule synthesis system according to claim 1, further comprising at least one nucleic acid template selected from the group consisting of a DNA template and an RNA template.

39. The in vitro protein or biological macromolecule synthesis system according to claim 1, further comprising components selected from the group consisting of:
at least one extract from a cell having reduced activity of at least one enzyme that catalyzes hydrolysis of high-energy phosphate bonds,
at least one extract from a cell having reduced activity of at least one enzyme that catalyzes hydrolysis or formation of phosphodiester bonds,
at least one inhibitor of at least one enzyme that catalyzes hydrolysis of high-energy phosphate bonds, and
at least one inhibitor of at least one enzyme that catalyzes hydrolysis or formation of phosphodiester bonds.

40. The in vitro protein or biological macromolecule synthesis system according to claim 1, further comprising components selected from the group consisting of a protease inhibitor and a phosphatase inhibitor.

41. The in vitro protein or biological macromolecule synthesis system according to claim 1, further comprising:
one or more nucleic acid templates, and
components selected from the group consisting of, at least one inhibitor of an enzyme that degrades said template, at least one extract from a cell having reduced degradative effect on said template, and at least one enzyme with reduced activity to catalyze hydrolysis or formation of phosphodiester bonds.

42. The in vitro protein or biological macromolecule synthesis system according to claim 1, further comprising a molecular chaperone or a foldase.

43. The in vitro protein or biological macromolecule synthesis system according to claim 42, wherein said molecular chaperone or foldase is selected from the group consisting of GroEL/ES, GroEL, GroES, TF, DnaK, DnaJ, GrpE, ClpB, FkpA, Skp, Dsb, DsbC, peptidyl prolyl cis/trans isomerase (PPI), chaperonin 60, chaperonin 10, TCP1, TF55, heat shock protein 60, Cpn60, heat shock protein 10, Cpn10, Lim protein, and signal recognition particle.

44. The in vitro protein or biological macromolecule synthesis system according to claim 43, wherein said molecular chaperone or foldase comprises GroEL/ES, TF, DnaK, DnaJ, GrpE, ClpB, FkpA, Skp and DsbC.

45. The in vitro protein or biological macromolecule synthesis system according to claim 1, wherein said synthesis of biological macromolecule comprises translation of mRNA to produce polypeptides or protein.

46. The in vitro protein or biological macromolecule synthesis system according to claim 45, wherein said synthesis of biological macromolecule comprises transcription from a DNA template to produce mRNA.

47. The in vitro protein or biological macromolecule synthesis system according to claim 1, wherein said glycolytic intermediate is present at an initial concentration of at least about 1 mM.

48. The in vitro protein or biological macromolecule synthesis system according to claim 1, wherein said glycolytic intermediate is present at an initial concentration of at least about 10 mM.

49. A composition for in vitro protein or biological macromolecule synthesis comprising one or more energy sources providing chemical energy for protein or biological macromolecule synthesis wherein at least one of said energy sources is a glycolytic intermediate.

50. The composition of claim 49, wherein said glycolytic intermediate is selected from the group consisting of 3-phosphoglycerate, 2-phosphoglycerate, 2,3,-diphosphoglycerate and 1,3-diphosphoglycerate.

51. The composition of claim 49, wherein said glycolytic intermediate is 3-phosphoglycerate.

52. The composition of claim 49, wherein said glycolytic intermediate provides chemical energy for protein or biological macromolecule synthesis without addition of an exogenous enzymatic cofactor.

53. The composition of claim 52, wherein said exogenous enzymatic cofactor is selected from the group consisting of thiamine pyrophosphate, FAD⁺, FADH, NAD⁺, NADH, NADP⁺ and NADPH.

54. The composition of claim 52, wherein said exogenous enzymatic cofactor is selected from the group consisting of NAD⁺ and NADH.

55. The composition of claim 49, further comprising a nucleic acid template selected from the group consisting of a DNA template and an RNA template.

56. The composition of claim 49, further comprising a cell extract to provide components selected from the group consisting of enzymes, ribosomes, transcription factors, translation factors and co-factors.

57. The composition of claim 49, wherein said glycolytic intermediate provides chemical energy for protein or biological macromolecule synthesis without addition of an exogenous enzyme.

58. The composition of claim 57 wherein said exogenous enzyme is selected from the group consisting of pyruvate kinase, acetate kinase, pyruvate oxidase, creatine kinase and creatine phosphokinase.

59. The composition of claim 49, wherein said one or more energy sources further comprises a tricarboxylic acid (TCA) cycle intermediate.

60. The composition of claim 59, wherein said TCA cycle intermediate is citrate, isocitrate, malate, oxaloacetate, α-ketoglutarate, succinate or fumarate.

61. The composition of claim 60, wherein said TCA cycle intermediate is citrate.

62. The composition of claim 59, wherein said TCA cycle intermediate and said glycolytic intermediate further comprise an exogenous enzymatic cofactor to generate or regenerate high-energy triphosphate compounds for protein or biological macromolecule synthesis.

63. The composition of claim 62, wherein said exogenous enzymatic cofactor is selected from the group consisting of thiamine pyrophosphate, FAD⁺, FADH, NAD⁺, NADH, NADP⁺ and NADPH.

64. The composition of claim 63, wherein said exogenous enzymatic cofactor is selected from the group consisting of NAD⁺ and NADH.

65. A kit for in vitro protein or biological macromolecule synthesis comprising a nucleic acid template, a cell extract, and one or more energy sources providing chemical energy for protein or biological macromolecule synthesis wherein at least one of said energy sources is a glycolytic intermediate.

66. The kit of claim 65 wherein said glycolytic intermediate is selected from the group consisting of 3-phosphoglycerate, 2-phosphoglycerate, 2,3,-diphosphoglycerate and 1,3-diphosphoglycerate.

67. The kit of claim 65 wherein said glycolytic intermediate is 3-phosphoglycerate.

68. The kit of claim 65 wherein said glycolytic intermediate provides chemical energy for protein or biological macromolecule synthesis without addition of an exogenous enzymatic cofactor.

69. The kit of claim 68, wherein said exogenous enzymatic cofactor is selected from the group consisting of thiamine pyrophosphate, FAD⁺, FADH, NAD⁺, NADH, NADP⁺ and NADPH.

70. The kit of claim 68 wherein said exogenous enzymatic cofactor is selected from the group consisting of NAD⁺ and NADH.

71. The kit of claim 65, wherein said glycolytic intermediate provides chemical energy for protein or biological macromolecule synthesis without addition of an exogenous enzyme.

72. The kit of claim 71 wherein said exogenous enzyme is selected from the group consisting of pyruvate kinase, acetate kinase, pyruvate oxidase, creatine kinase, and creatine phosphokinase.

73. A method for producing protein or biological macromolecule from a nucleic acid template in an in vitro protein or biological macromolecule synthesis system comprising:
contacting said nucleic acid template with one or more energy sources providing chemical energy for protein or biological macromolecule synthesis to form a mixture wherein at least one of said energy sources is a glycolytic intermediate;
contacting said nucleic acid template and said one or more energy sources with a cell extract; and
incubating said mixture under conditions sufficient to produce at least one protein or biological macromolecule encoded by said template.

74. The method of claim 73 wherein said glycolytic intermediate is selected from the group consisting of 3-phosphoglycerate, 2-phosphoglycerate, 2,3,-diphosphoglycerate and 1,3-diphosphoglycerate.

75. The method of claim 73 wherein said glycolytic intermediate is 3-phosphoglycerate.

76. The method of claim 73 wherein said glycolytic intermediate provides chemical energy for protein or biological macromolecule synthesis without addition of an exogenous enzymatic cofactor.

77. The method of claim 76 wherein said exogenous enzymatic cofactor is selected from the group consisting of thiamine pyrophosphate, FAD⁺, FADH, NAD⁺, NADH, NADP⁺ and NADPH.

78. The method of claim 76 wherein said exogenous enzymatic cofactor is selected from the group consisting of NAD⁺ and NADH.

79. The method of claim 73, wherein said glycolytic intermediate provides chemical energy for protein or biological macromolecule synthesis without addition of an exogenous enzyme.

80. The method of claim 73, wherein said incubating said mixture under conditions sufficient to produce at least one protein or biological macromolecule encoded by said template is performed as a batch reaction.

81. The method of claim 73, wherein said incubating said mixture under conditions sufficient to produce at least one protein or biological macromolecule encoded by said template is performed as a continuous reaction.

82. A method for constructing an in vitro protein or biological macromolecule synthesis system, said method comprising:
contacting a nucleic acid template with at least one cell extract; and
contacting one or more energy sources providing chemical energy for protein or biological macromolecule synthesis with said nucleic acid template and said at least one cell extract wherein at least one of said energy sources is a glycolytic intermediate.

83. The method of claim 82 wherein said glycolytic intermediate is selected from the group consisting of 3-phosphoglycerate, 2-phosphoglycerate, 2,3,-diphosphoglycerate and 1,3-diphosphoglycerate.

84. The method of claim 82 wherein said glycolytic intermediate is 3-phosphoglycerate.

85. The method of claim 82 wherein said glycolytic intermediate provides chemical energy for protein or biological macromolecule synthesis without addition of an exogenous enzymatic cofactor.

86. The method of claim 85, wherein said exogenous enzymatic cofactor is selected from the group consisting of thiamine pyrophosphate, FAD⁺, FADH, NAD⁺, NADH, NADP⁺ and NADPH.

87. The method of claim 85 wherein said exogenous enzymatic cofactor is selected from the group consisting of NAD⁺ and NADH.

88. The method of claim 82 wherein said glycolytic intermediate provides chemical energy for protein or biological macromolecule synthesis without addition of an exogenous enzyme.

89. A composition for in vitro protein or biological, macromolecule synthesis comprising:
one or more energy sources providing chemical energy for protein or biological macromolecule synthesis wherein at least one of said energy sources is a glycolytic intermediate;
further comprising components selected from the group consisting of:
at least one extract from a cell having reduced activity of at least one enzyme that catalyzes hydrolysis of high-energy phosphate bonds or hydrolysis or formation of phosphodiester bonds;
at least one inhibitor of at least one enzyme that catalyzes hydrolysis of high-energy phosphate bonds or hydrolysis or formation of phosphodiester bonds;
at least one inhibitor of an enzyme that degrades said template; and
at least one extract from a cell having reduced degradative effect on said template.

90. The composition of claim 89 wherein said glycolytic intermediate is selected from the group consisting of 3-phosphoglycerate, 2-phosphoglycerate, 2,3,-diphosphoglycerate and 1,3-diphosphoglycerate.

91. The composition of claim 89 wherein said glycolytic intermediate is 3-phosphoglycerate.

92. The composition of claim 89 wherein said glycolytic intermediate provides chemical energy for protein or biological macromolecule synthesis without addition of an exogenous enzymatic cofactor.

93. The composition of claim 92, wherein said exogenous enzymatic cofactor is selected from the group consisting of thiamine pyrophosphate, FAD⁺, FADH, NAD⁺, NADH, NADP⁺ and NADPH.

94. The composition of claim 92 wherein said exogenous enzymatic cofactor is selected from the group consisting of NAD⁺ and NADH.

95. The composition of claim 89, further comprising adenosine triphosphate and cysteine.

96. The composition of claim 89, wherein said glycolytic intermediate provides chemical energy for protein or biological macromolecule synthesis without addition of an exogenous enzyme.
